(19) United States
(12) Patent Application Publication
(10) Pub. No.: US 2004/0219619 A1
(43) Pub. Date: Nov. 4, 2004

(54) Methods of identifying compounds that alter toxin persistence and/or protease activity

(76) Inventors: Ester Fernandez-Salas, Fullerton, CA (US); Lance E. Steward, Irvine, CA (US); Joseph Francis, Aliso Viejo, CA (US); Kei Roger Aoki, Coto de Caza, CA (US)

Correspondence Address: Quan L. Nguyen, Cozen O'Connor, 1900 Market Street, Philadelphia, PA, 19103, US

(21) Appl. No.: 10/759,746

(22) Filed: Jan. 16, 2004

Related U.S. Application Data
(63) Continuation-in-part of application No. 10/163,106, filed on Jun. 4, 2002.
Application No. 10/163,106 is a continuation-in-part of application No. 09/910,346, filed on Jul. 20, 2001.
Application No. 09/910,346 is a continuation-in-part of application No. 09/620,840, filed on Jul. 21, 2000.

(51) Int. Cl. G01N 33/554; G01N 33/569; A61K 39/08

(52) U.S. Cl. 435/7.32; 424/239.1

(57) ABSTRACT

Methods of identifying compounds that inhibit the biological persistence of a Clostridial toxin are disclosed. Methods of identifying compounds that inhibit the enzymatic activity of Clostridial toxin are disclosed. Methods of identifying compounds that inhibit both the biological persistence and enzymatic activity of a Clostridial toxin are disclosed. Methods of identifying compounds that enhance the biological persistence of a Clostridial toxin are disclosed. Methods of identifying compounds that enhance the enzymatic activity of Clostridial toxin are disclosed. Methods of identifying compounds that enhance both the biological persistence and enzymatic activity of a Clostridial toxin are disclosed.

### CROSS-REFERENCE TO RELATED APPLICATIONS

This application is a continuation-in-part of U.S. application Ser. No. ______ [Attorney Docket No. ALLE0014-103] filed Jan. 14, 2004, which is a continuation-in-part of U.S. application Ser. No. 10/163,106 filed Jun. 4, 2002, which is a continuation-in-part of U.S. application Ser. No. 09/910,346 filed Jul. 20, 2001; which is a continuation-in-part U.S. application Ser. No. 09/620,840 filed Jul. 21, 2000, the disclosures of which are incorporated herein by reference in their entirety.

### FIELD OF THE INVENTION

The present invention is directed to identification of compounds and the development of new drug therapies for the treatment of botulism and tetanus. Additionally, the present invention is directed to identification of compounds that can interfere with or affect a biochemical pathway, such as by attenuating the signals involved in toxin light chain subcellular localization and trafficking and/or modulation of protease activity.

### BACKGROUND OF THE INVENTION

Clostridial neurotoxins produced by the anaerobic bacteria Clostridium botulinum and Clostridium tetani are some of the most potent naturally occurring compounds known and are the causative agents of botulism and tetanus, respectively. Botulinum neurotoxins (BoNTs) are typically encountered in food poisoning, although they also occur as a colonizing infection of the neonatal intestinal tract (infant botulism) and, rarely, as a result of wound infection (wound botulism). In 2001, 169 cases of botulism were reported to the U.S. Center for Disease Control (CDC). Of these, 33 were food-borne, 112 were infant botulism, and 23 were cases of wound botulism. Death can result from respiratory failure (in about 8% of botulism cases). Recovery can take months and those who survive may have fatigue and shortness of breath for years.

Botulism due to food poisoning is a rare occurrence in developed countries, but a significant number of cases occur each year from eating home canned foods. Infant botulism is the most common form of botulism in the USA, with 1134 cases reported to the CDC from 1976 through 1992. Supportive care is the most common treatment and usually results in complete recovery after several weeks. If diagnosed early, patients may be treated with equine antitoxin serum that neutralizes circulating toxin and, therefore, minimizes subsequent nerve damage. The equine antitoxin treatment will not, however, reverse existing paralysis due to toxin already internalized in neurons (Arnon, et al., Botulinum toxin as a biological weapon: medical and public health management. (2001) JAMA 285:1059-1070). A few years ago, a human hyperimmune serum (Botulism Immune Globulin Intravenous) was developed to treat infant botulism (Arnon et al., supra).

Tetanus toxin (TeNT) is encountered as a wound contaminant and is a health problem in developing countries. Tetanus is controlled in developed countries via childhood vaccination.

A significant outbreak of botulism can entail a need for mechanical ventilators, critical care beds, and skilled personnel that can exceed capacity. Additional research into diagnosis and treatment of botulism is therefore needed.

Botulinum toxins have been used in clinical settings for the treatment of neuromuscular disorders characterized by hyperactive skeletal muscles. In 1989 a botulinum toxin type A complex was approved by the U.S. Food and Drug Administration for the treatment of blepharospasm, strabismus and hemifacial spasm. Subsequently, a botulinum toxin type A was also approved by the FDA for the treatment of cervical dystonia and for the treatment of glabellar lines, and a botulinum toxin type B was approved for the treatment of cervical dystonia. Non-type A botulinum toxin serotypes apparently have a lower potency and/or a shorter duration of activity as compared to botulinum toxin type A. Clinical effects of peripheral intramuscular botulinum toxin type A are usually seen within one week of injection. The typical duration of symptomatic relief from a single intramuscular injection of botulinum toxin type A averages about three months, although significantly longer periods of therapeutic activity have been reported.

It has been reported that botulinum toxin type A has been used in clinical settings to treat constipation by intrasphincter injection of the puborectalis muscle; to treat upper limb spasticity following stroke; and to treat migraines. Additionally, intramuscular botulinum toxin has been used in the treatment of tremor in patient's with Parkinson's disease.

It is known that botulinum toxin type A can have an efficacy for up to 12 months (European J. Neurology 6 (Supp 4): S111-S1150:1999), and in some circumstances for as long as 27 months (The Laryngoscope 109:1344-1346:1999). However, the usual duration of an intramuscular injection is typically about 3 to 4 months.

The success of botulinum toxin type A to treat a variety of clinical conditions has led to interest in other botulinum toxin serotypes. Two commercially available botulinum type A preparations for use in humans are BOTOX® available from Allergan, Inc., of Irvine, Calif., and Dysport® available from Beaufour Ipsen, Porton Down, England. A Botulinum toxin type B preparation (MyoBloc®) is available from Elan Pharmaceuticals of San Francisco, Calif.

In addition to having pharmacologic actions at the peripheral location, botulinum toxins may also have inhibitory effects in the central nervous system.

A botulinum toxin has also been proposed for the treatment of rhinorrhea, hyperhydrosis and other disorders mediated by the autonomic nervous system (U.S. Pat. No. 5,766,605), tension headache, (U.S. Pat. No. 6,458,365), migraine headache (U.S. Pat. No. 5,714,468), post-operative pain and visceral pain (U.S. Pat. No. 6,464,986), pain treatment by intraspinal toxin administration (U.S. Pat. No. 6,113,915), Parkinson's disease and other diseases with a motor disorder component, by intracranial toxin administration (U.S. Pat. No. 6,306,403), hair growth and hair retention (U.S. Pat. No. 6,299,893), psoriasis and dermatitis (U.S. Pat. No. 5,670,484), injured muscles (U.S. Pat. No. 6,423,319, various cancers (U.S. Pat. Nos. 6,139,845), pancreatic disorders (U.S. Pat. No. 6,143,306), smooth muscle disorders (U.S. Pat. No. 5,437,291, including injection of a botulinum toxin into the upper and lower esophageal, pyloric and anal sphincters), prostate disorders (U.S. Pat. No. 6,365,164), inflammation, arthritis and gout (U.S. Pat. No. 6,063,768), juvenile cerebral palsy (U.S. Pat. No. 6,395,277), inner ear disorders (U.S. Pat. No. 6,265,379), thyroid disorders (U.S. Pat. No. 6,358,513), and parathyroid disorders (U.S. Pat. No. 6,328,977). Additionally, controlled release toxin implants are known (see e.g. U.S. Pat. Nos. 6,306,423 and 6,312,708).

Seven generally immunologically distinct botulinum neurotoxins have been characterized: botulinum neurotoxin serotypes A, B, C₁, D, E, F and G. These serotypes are distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg, which is about 12 times the primate LD₅₀ for botulinum toxin type A (Moyer E et al., Botulinum Toxin Type B. Experimental and Clinical Experience, being chapter 6, pages 71-85 of “Therapy With Botulinum Toxin”, edited by Jankovic, J. et al. (1994), Marcel Dekker, Inc.). Botulinum toxin apparently binds with high affinity to cholinergic motor neurons, is translocated into the neuron and blocks the release of acetylcholine.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain (H chain) and a cell surface receptor and/or specific ganglioside; the receptor and/or ganglioside is thought to be different for each type of botulinum toxin and for tetanus toxin. In the H chain, a carboxyl end segment, known as H_{c}, appears to be a receptor binding domain important for targeting of the toxin to the cell surface.

In the second step, the toxin is internalized into the cell. The toxin is first engulfed by the cell through receptor-mediated endocytosis, and an endosome containing the toxin is formed. The toxin then translocates from the endosome into the cytoplasm of the cell. This step is thought to be mediated by an amino terminal domain of the H chain, H_{N}, which undergoes a conformational change in response to a pH of about 5.5 or lower. Endosomes are known to possess a proton pump that decreases intra-endosomal pH. The conformational shift exposes hydrophobic residues in the toxin, which permits the toxin to embed itself in the endosomal membrane. The toxin (or at a minimum the light chain) then translocates through the endosomal membrane into the cytoplasm.

The last step of the mechanism of botulinum toxin activity appears to involve reduction of the disulfide bond joining the heavy chain (H chain or HC) and the light chain (L chain or LC). The entire toxic activity of botulinum and tetanus toxins is contained in the L chain of the holotoxin. The L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane.

Although all the botulinum toxins serotypes apparently inhibit release of the neurotransmitter acetylcholine at the neuromuscular junction, they do so by affecting different neurosecretory proteins and/or cleaving these proteins at different sites. Each of the botulinum toxins specifically cleaves a different bond. Botulinum types A and E both cleave the 25 kiloDalton (kD) synaptosomal associated protein (SNAP-25), but they target different amino acid sequences within this protein. Botulinum toxin types B, D, F and G and tetanus toxin act on vesicle-associated membrane protein (VAMP, also called synaptobrevin), and/or cellubrevin (V-SNARE present in non-neuronal cells), with each serotype of Botulinim toxin cleaving the protein at a different site. Most of the VAMP present at the cytoplasmic surface of the synaptic vesicle is removed as a result of any one of these cleavage events. Botulinum toxin type B and tetanus toxin cleave the same bond of VAMP. Finally, botulinum toxin type C₁ ^{has been shown to cleave both syntaxin and SNAP-}25. These differences in mechanism of action may affect the relative potency and/or duration of action of the various botulinum toxin serotypes. Apparently, a substrate for a botulinum toxin can be found in a variety of different cell types (See e.g. Biochem, J 1;339 (pt 1):159-65:1999, and Mov Disord, 10(3):376:1995 (pancreatic islet B cells contains at least SNAP-25 and synaptobrevin)).

The subcellular localization patterns of the L chains from Botulinum toxin types A, B and E are distinctly different, with LC/A localizing to the plasma membrane, LC/E localizing to the cytoplasm (with nuclear exclusion), and LC/B being dispersed throughout the cell (including the nucleus) (Fernandez-Salas E, et al. Localization of BoNT light chains in neuronal and non-neuronal cell lines, implications for the duration of action of the different serotypes. Naunyn-schmiedebergs archives of pharmacology 365: 144 Suppl. 2, 2002). In some embodiments, inactive LC/A has been found to localized to the plasma membrane. The duration of action of the neurotoxins BoNT/A>BoNT/B>>BoNT/E is believed to be related to their subcellular localization. The LC of BoNT/A, with the longest duration of effect, localizes to the plasma membrane, a compartment known for its low rate of protein recycling. This specific localization may protect LC/A from proteolytic degradation within neurons, while the shorter duration of LC/E may be related to cytoplasmic localization and increased susceptibility to degradation. Moreover, the sequences and motifs in LC/A that are important for plasma membrane localization have been identified. Mutations or truncations of these sequences result in a protein with different subcellular localization and diminished activity with respect to the SNAP25 substrate (Steward LE, et al., BoNT/A light chain and the dileucine motif: Potential implications for light chain localization and neurotoxin duration of action. Naunyn-schmiedebergs archives of pharmacology 365:144 Suppl. 2, 2002).

Currently, no small-molecule approved drugs are available that prevent the appearance of symptoms following exposure and acute intoxication with botulinurn toxins. The muscular paralysis elicited by BoNTs is due to the cleavage of one or more of the SNARE proteins (SNAP25, VAMP, and syntaxin) essential for vesicle docking and fusion at the nerve terminal. The endopeptidase activity of the neurotoxin is contained within the light chain (LC), which is believed to reside in the neurons for long periods of time for some of the serotypes.

Efforts have been made to develop small-molecule inhibitors of BoNTs that target the enzymatic active site of the various serotypes, thereby preventing the cleavage of the respective SNARE protein targets. A crystal structure of BoNT/B with a metalloprotease inhibitor (BABIM) has been recently reported (Eswaramoorthy, S., et al., A novel mechanism for Clostridium botulinum neurotoxin inhibition (2002) Biochemistry 41: 9795-9802) but has not been tested in any cellular models. The ability of 3,4-diaminopyridine (3,4-DAP) to antagonize muscle paralysis following local injection of botulinurn neurotoxin A (BoNT/A) complex has been evaluated in the in situ rat extensor digitorum longus (EDL) preparation. 3,4-DAP reduced paralysis only with sustained delivery with functional impairment returning to levels observed in control (untreated) animals upon drug treatment cessation (Adler M, et al., Antagonism of botulinum toxin A-mediated muscle paralysis by 3,4-diaminopyridine delivered via osmotic minipumps (2000). Toxicon 38:1381-8).

Although it is important to identify compounds that are capable of inhibiting the biological persistence of Clostridial neurotoxins, it is also important to be able to identify compounds that increase the biological persistence of Clostridial neurotoxins because the clinical benefit for the patient will be extended several months, allowing for fewer injections per year, and fewer visits to the doctor's office. Thus, it would be advantageous to be able to identify compounds that increase the biological persistence of, for example, Botulinum toxin types B and E.

There remains a need to find compounds that alter the biological persistence in Botulinum toxin. There remains a need to find compounds that neutralize or diminish the biological persistence of Clostridial neurotoxins. There remains a need to find compounds that can be useful to treat botulism and/or tetanus intoxication. There remains a need to find compounds that can be useful to neutralize or diminish the biological persistence Clostridial neurotoxins and thereby lessen their effectiveness as bioterrorism and biological warfare weapons. There remains a need to find compounds that can be useful to neutralize or diminish the biological persistence of the Clostridial neurotoxins in the event that reversal of their effects is desired in an individual after therapeutic application. There remains a need to find compounds that can be useful to increase the biological persistence and/or biological activity of Clostridial neurotoxins. Such compounds with increased biological persistence may allow a longer time between dosings. Such compounds with increased activity may allow a reduction in the dosage given. Both kinds of compounds are expected to result in fewer visits to the doctor and a reduction of the risk of antibody formation.

### SUMMARY OF THE INVENTION

The present invention provides methods of identifying compounds that alters, i.e. inhibit or enhances, the biological persistence of a Clostridial toxin. The methods comprise performing a test localization assay that comprises the steps of contacting a cell that comprises a Clostridial toxin light chain with a test compound and determining whether the localization pattern of the light chain in the cell differs following contacting the cell with the test compound compared to the localization pattern of the light chain in the cell in the absence of the test compound. A change in the localization pattern of the light chain in the cell following contacting the cell with the test compound indicates that the test compound alters, i.e., inhibits or enhances, the biological persistence of the toxin.

Without wishing to limit the invention to any theory or mechanism of operation, it is believed that the disruption of the localization pattern of Botulinum toxin type A from the plasma membrane decreases biological persistence. In some embodiments, the biological persistence of Botulinum toxin type A may be decreased by a test compound. In some embodiments, a test compound that decreases the biological persistence of Botulinum toxin type A is capable of disrupting the localization pattern of type A. For example, a compound that decreases the biological persistence of Botulinum toxin type A causes the light chain of Botulinum toxin type A to no longer localize at the plasma membrane. In some embodiments, a compound that decreases biological persistence of Botulinum toxin type A causes the light chain of type A to be dispersed throughout the cell.

Without wishing to limit the invention to any theory or mechanism of operation, it is believed that the localization of Botulinum toxin to the plasma membrane similarly to that of type A enhances biological persistence. In some embodiments, the biological persistence of Botulinum toxin types B and E may be increased by a test compound. In some embodiments, a test compound is capable of causing the Botulinum toxin type B to localize to the appropriate cellular membrane or organelle, reducing the rate of degradation of the protein and extending its half-life. In some embodiments, a test compound is capable of causing the Botulinum toxin type E to localize to the appropriate cellular membrane or organelle, reducing the rate of degradation of the protein and extending its half-life.

The present invention relates to methods of identifying compounds that alter, i.e., inhibit or enhance, the enzymatic activity of Clostridial toxin. The methods comprise performing a test enzymatic assay that comprises contacting a sample containing the light chain of the toxin with an enzymatic substrate of the light chain in the presence of the test compound, and determining whether the substrate is processed by the light chain into enzymatic product. The absence of processing of the enzymatic substrate into enzymatic product indicates that the test compound inhibits enzymatic activity. The increase of processing of enzymatic substrate into enzymatic product indicates that the test compound enhances enzymatic activity.

The present invention relates to methods of identifying compounds that alter, i.e., enhance or inhibit, the biological persistence and enzymatic activity of a Clostridial toxin. The methods comprise performing both a test localization assay and a test enzymatic assay using a test compound. The assays may be run in either order or simultaneously.

### BRIEF DESCRIPTION OF THE DRAWINGS

FIG. 1 shows data from experiments described in Example 3. The localization of the GFP-LC fusion proteins having GFP fused to light chain A, B or E in differentiated PC12 cells (plus control GFP only). The images are confocal images with the focal point in the middle of the cell.

FIG. 2 shows data from experiments described in Example 3. The localization of the GFP-LC fusion proteins having GFP fused to light chain A, B or E in the human neuroblastoma cell line, SH-SY5Y (plus control GFP only).

FIGS. 3A-3D show data from experiments described in Example 4. Subcellular localization of the LC/A mutants were detected using confocal microscopy. FIG. 3A shows the localization pattern of GFP-LC/A (ΔN8ΔC22), FIG. 3B shows the localization pattern of GFP-LC/A(AA), FIG. 3C shows the localization pattern of GFP-LC/A(ΔC22), and FIG. 3D shows the localization pattern of GFP-LC/A(ΔN8).

FIG. 4 shows data from Example 5 in which Western Blots of PC12 cell lysates transfected with the G-FP-LC/A mutants were performed. Blots were probed with an antibody that recognizes specifically the cleaved product of LC/A, SNAP25₁₉₇. Equal amounts of cell lysates were loaded on each lane. LCAAG: LC/A(AA)-GFP, GLCAA: GFP-LC/A(AA), tLCAG: LC/A(ΔN8/ΔC22)-GFP, GtLCA: GFP-LC/A(ΔN8/ΔC22), LCAG:LC/A-GFP, GLCA:GFP-LC/A.

FIG. 5 shows data from Example 5 in which an analysis of the catalytic activity of rLC/A mutants was done. Activity of purified wild-type rLC/A and rLC/A mutants in the SNAP25 ELISA assay was determined. Plates coated with SNAP25 substrate were incubated with varying concentrations of BoNT/A, rLC/A or rLC/A mutants as indicated. Cleavage was detected with an antibody specific for SNAP25₁₉₇. Wild-type rLC/A has an effective concentration (EC₅₀) of 7 pM, and its catalytic activity is unaffected by the position of the His-tag. rLC/A(AA) has an EC₅₀ of 184 pM and is 26-times less active than wild-type upon the SNAP25 substrate. rLC/A(AC22) has an EC₅₀ of 566 pM and is 81-times less active than wild-type. rLC/A(ΔN8/ΔC22) has an EC₅₀ of 4663 pM and is 666-times less active than wild type. With respect to localization, rLC/A localizes to the plasma membrane, regardless of the GFP-tag position. rLC/A(AA) and rLC/A(AC22) diffuse throughout the plasma membrane. rLC/A(ΔN8) is mainly in the cytoplasm and rLC/A(ΔN8/ΔC22) is dispersed intracellularly.

FIG. 6A shows schematic representations of adaptins (AP) proteins and the VHS subunit of GGAS. FIG. 6B show the localization and function of adaptins. These proteins are involved in intracellular trafficking and are potential drug targets.

### DESCRIPTION OF PREFERRED EMBODIMENTS

Definitions

As used herein, the term “biological persistence” is meant to refer to the continuous period of time that a light chain retains its enzymatic activity when that light chain is within a cell or outside of a cell.

As used herein, the term “light chain” is meant to refer to a protein that comprises the amino acid sequence of the light chain endopeptidase of a Clostridial toxin, or a modified light chain in which certain amino acids have been mutated or deleted.

As used herein, the term “localization pattern” is meant to refer to the intracellular localization of the light chain. Light chains from different serotypes display different localization patterns, i.e. the light chains of different serotypes are predominantly found in a particular locations within the cell based upon the serotype of toxin. For example, the localization pattern of light chain A is displayed as the light chain A localizing to the plasma membrane. The localization pattern of light chain E is displayed as the light chain E localizing to the cytoplasm with nuclear exclusion. The localization pattern of light chain B is displayed as the light chain B being dispersed throughout the cell including the nucleus.

As used herein, the term “localization assay negative control compound” is meant to refer a compound that is known to have no effect on the localization pattern of a light chain in a cell when contacted with a cell that comprises the light chain.

As used herein, the term “localization assay positive control compound” is meant to refer to a compound that is known to change the localization pattern of a light chain in a cell when contacted with a cell that comprises the light chain.

As used herein, the term “enzymatic assay negative control compound” is meant to refer to a compound that is known not to inhibit the enzymatic activity of a light chain in an assay using that light chain with an enzymatic substrate known to be otherwise processed by the light chain into enzymatic products.

As used herein, the term “enzymatic assay positive control compound” is meant to refer to a compound that is known to inhibit the enzymatic activity of a light chain in an assay using that light chain with an enzymatic substrate known to be otherwise processed by the light chain into enzymatic products. Examples of enzymatic assay positive control compound include 3,4-diaminopyridine, and zinc chelators (e.g., ethylene diamine tetraacetate, (EDTA), tetrakis(2-pyridylmethyl)ethylenediamine (TPEN), o-phenanthroline; BoNT/A inhibitors include Ac-CRATKML-NH-2(Schmidt et al. 1988, FEBS Lett 4356,61-64); BoNT/B BABIM inhibitor (M. A. Hanson et al. 2000, J. Am. Chem. Soc. 122, 11268); the disclosures of which are incorporated in their entirety herein by reference.

As used herein, the term “enzymatic substrate” is meant to refer to a protein, peptide or peptidomimetic that can be processed, i.e., cleaved, by a light chain. The different light chain serotypes have various naturally occurring enzymatic substrates. Botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Tetanus toxin cleaves VAMP at the same site that Botulinum toxin type B cleaves VAMP. Finally, botulinum toxin type C₁ has been shown to cleave both syntaxin and SNAP-25.

As used herein, the term “enzymatic product” is meant to refer to the protein, peptides or peptidomimetic sub-units formed upon cleavage of the enzymatic substrate by the light chain. In addition, enzymatic product is also intended to refer to, in addition to the protein sub-units formed upon cleavage of the enzymatic substrate by the light chain, other detectable products which are formed either due to the processing of the enzymatic substrate by the light chain or as products of subsequent reactions that occur only after processing of the enzymatic substrate by the light chain.

Overview

The present invention provides methods of identifying compounds that are useful to alter, e.g., eliminate/reduce or enhance/increase, the biological persistence of toxins produced by species of the prokaryotic genus Clostridial. In particular, methods are provided for identifying compounds that alter, i.e. inhibit or enhance, the biological persistence of the toxins and/or the enzymatic activity of the toxin.

The toxins are heterodimers having a light chain and a heavy chain. The biological activities of the toxins reside in the light chain. Accordingly, the present invention provides methods of identifying compounds that are useful to alter, e.g., eliminate/reduce or enhance/increase, the biological persistence associated with the light chain of the toxins.

According to one aspect of the invention, interference with the localization and trafficking signals in the light chain A and other botulinum toxin serotypes or tetanus toxin, will affect the trafficking, localization, duration and/or activity of the neurotoxin. See, for example, U.S. patent application Ser. Nos. 09/620,840 to Steward et al; 09/910,346 to Steward et al. and 10/163,106 to Steward et al., commonly assigned to Allergan, Inc., Irvine, Calif., USA, the disclosures of which are incorporated in their entirety herein by reference. Thus, modulation of the light chain will render a protein that is either degraded or mislocalized due to improper trafficking, has lower activity and/or shorter duration of action. Compounds such as small molecule drugs, peptides or peptidomimetics that will interact with such signals or sequences that either inactivate the light chain endopeptidase activity, remove the light chain from the normal localization compartment so it will be easily degraded in the cell, or rerouted to lysosomes for degradation upon uptake are identified. Such molecules will be able to bind toxin in circulation, interfere with the light chain while trafficking in the neuron, displace light chain from its subcellular compartment, and/or reduce the catalytic activity of the endopeptidase.

In some embodiments, molecules that interfere with enzyme activity are identified. Such molecules include molecules that bind to areas remote from the active site and have an inhibitory effect on the catalytic activity allosterically. Such inhibition of enzyme activity may be totally independent of changes in localization, or have dual activity.

It has been observed that truncated or mutated recombinant light chain A have reduced catalytic activity when tested in an in vitro ELISA assay using purified protein and purified substrate. This reduction of activity can also be seen in cell lysates having the native SNAP25 present in the cell. The loss of the N-terminus, for example, has a dramatic effect on catalytic activity. In some embodiments, compounds are screened to identify compounds that interact with the N-terminus and are complete or partial inhibitors of the catalytic activity. The loss of a di-leucine motif found within the last 22 amino acids of the C-terminus of the light chain, for example, can also affect catalytic activity. In some embodiments, compounds are screened to identify compounds that interact with the C-terminus and are complete or partial inhibitors of the catalytic activity. In addition, molecules binding to other areas in the light chain may provide a similar allosteric effect on activity with or without an effect on localization.

It has been observed that mutations or truncations of the light chain A have a profound effect on subcellular localization. These mutated forms of light chain A are believed to have a shorter duration of action in neurons due to greater presentation to cellular degradation pathways. Small molecule inhibitors may have an effect similar to these truncations and mutations by interfering with protein-protein interactions between light chain and cellular proteins responsible for trafficking and subcellular localization. These inhibitors may render a protein with shorter half-life with or without an effect on catalytic activity.

Results demonstrate that mutations and truncations to light chain A have a dramatic effect on localization as well as a dramatic effect on enzymatic activity, when tested in cell lysates or in the in vitro ELISA assay. Inhibitors that affect both localization and enzymatic activity and thereby render a protein that is less active and that is degraded faster may be identified according to the invention. It has been shown that inhibitors of enzymatic activity required sustained delivery after acute exposure to toxin to reverse paralysis (Adler et al., supra). These symptoms can last months for serotype A. Inhibitors of both localization and catalytic activity will present the advantage of immediate relief of symptoms through inhibition of the protease activity, and shorter half-life of the LC allowing for a faster, full recovery of the patient.

Localization Pattern

According to some aspects of the invention, compounds are identified that eliminate or reduce the biological persistence of the light chain of the toxin. The activity is eliminated or reduced by exposing cells that comprise the light chain of toxin with a compound that results in the toxin being more susceptible to degradation. The compounds act by disrupting the intracellular localization of the toxin light chain and thereby resulting in a reduction of the biological persistence of the light chain. By reducing the time that the light chain is an enzymatically active protein and facilitating the elimination of the enzymatically active protein the compound reduces the action of the light chain in the cell. For example, the light chain of Botulinum toxin type A generally localizes to the plasma membrane. A compound that is capable of disrupting the localization of the light chain of Botulinum toxin type A to the plasma membrane would be capable of eliminating or reducing the biological persistence of the light chain of the Botulinum toxin type A.

According to some aspects of the invention, compounds are identified that enhance or increase the biological persistence of the light chain of the toxin. The activity is enhanced or increased by exposing cells that comprise the light chain of toxin with a compound that results in the toxin being less susceptible to degradation. The compounds act by enhancing the membrane localization of the toxin light chain and thereby resulting in an increase of the biological persistence of the light chain. By increasing the time that the light chain is an enzymatically active protein increases the duration of action of the light chain in the cell. For example, the light chain of Botulinum toxin type E has fairly low biological persistence relative to type A. Botulinum toxin type E generally localizes to the cytoplasm of the cell. A compound that is capable of causing the light chain of Botulinum toxin type E to localize to the plasma membrane, similarly to the pattern of type A, would be capable of enhancing or increasing the biological persistence of the light chain of the Botulinum toxin type E.

According to some aspects of the invention, compounds are identified that inhibit or enhance the enzyme activity of the light chain of the toxin. The inhibition of the enzyme activity results in a reduction or neutralization; the enhancement results in an increase of the toxin's effect.

According to some aspects of the invention compounds are screened to determine if they either eliminate or reduce the biological persistence of the light chain of the toxin by disrupting the intracellular localization of the toxin light chain as well as by directly inhibiting or modulating the enzyme activity. In some embodiments, compounds are screened to determine if they increase or enhance the biological persistence of the light chain of the toxin by localizing the light chain of the toxin to the plasma membrane, or if they directly enhance the enzyme activity. In some embodiments, compounds are screened to determine if they both increase the biological persistence of the light chain of the toxin by localizing the light chain of the toxin to other membranes (e.g., ER membrane, endosomal membranes, Golgi membranes, trans Golgi network, etc.) as well as directly enhance the enzyme activity.

According to some embodiments, methods are provided for identifying a compound that alters, e.g., inhibits or enhances, the biological persistence of a Clostridial toxin. The methods comprise performing a test localization assay. In such an assay, a cell that comprises Clostridial toxin light chain is contacted with a test compound. Subsequently, the localization pattern of the light chain in the cell is detected and compared with the localization pattern of the light chain in the cell in the absence of the test compound to determine whether the localization pattern of the light chain has changed. A change in the localization pattern of the light chain in the cell following contacting the cell with the test compound indicates that the test compound will alter, i.e. inhibit or enhance, the biological persistence of the toxin.

In some embodiments, the localization pattern of the light chain in the cell is observed prior to contacting it with the test compound. In some embodiments, the localization pattern of the light chain in the cell in the absence of the test compound is based upon the predicted localization pattern such as that which has been observed in the same type of cell that has the light chain in it.

According to some embodiments, the Clostridial toxin is selected from the group consisting of: the toxin that is produced by Clostridium beratti, the toxin that is produced by Clostridium butyricum, the toxin that is produced by Clostridium tetani bacterium and the toxin that is produced by Clostridium botulinum. In some embodiments, the Clostridial toxin is selected from the group of Clostridial toxins consisting of: botulinum toxin types A, B, C₁, D, E, F and G.

According to some embodiments, the method further comprises performing a negative control localization assay. Such a negative control assay comprises contacting a cell that comprises the light chain with a localization assay negative control compound; and determining whether the localization pattern of the light chain in the cell differs following contacting the cell with the localization assay negative control compound compared to the localization pattern of the light chain in the cell in the absence of the localization assay negative control compound. A change in the localization pattern of the light chain in the cell in the negative control localization assay indicates that the test localization assay results are inconclusive.

According to some embodiments, the method further comprises performing a positive control localization assay. Such a positive control assay comprises contacting a cell that comprises the light chain with a localization assay positive control compound, and determining whether the localization pattern of the light chain in the cell differs following contacting the cell with the localization assay positive control compound compared to the localization pattern of the light chain in the cell in the absence of the localization assay positive control compound. An absence of change in the localization pattern of the light chain in the cell following contacting the cell with the localization assay positive control compound indicates that the test localization assay results are inconclusive.

According to some embodiments, multiple test localization assays are performed in which different concentrations of test compound are used in individual test assays. Generally, in such embodiments, the test compound is used in step-wise 10-fold dilutions to ascertain its activity and the concentration necessary to observe activity.

According to some embodiments, the method is performed using at least a duplicate (e.g., triplicate) test localization assays. By using duplicate assays, results are more reliable. In the event that one of the duplicate test assays is positive and the corresponding duplicate produces negative results, the assay is repeated.

According to some embodiments, the cell used in the assays is selected, for example, from the group consisting of: Neuro-2A cells, PC₁₂ cells, SH-SY5Y cells, HIT-T15 cells, HeLa cells, HEK293 cells, and other cell lines and primary cells from the central nervous system (e.g., spinal cord cells, dorsal root ganglion cells, cortex cells, and hippocampal cells).

According to some embodiments, the cell comprises a gene that encodes the light chain. When the gene is expressed, the light chain is produced in the cell.

According to some embodiments, the light chain is incorporated into the cell by contacting the cell with the toxin in an amount effective to be taken up by the cell and produce an identifiable localization pattern of the light chain in the cell.

According to some embodiments, the light chain is labeled. In some embodiments, the light chain is labeled with a radioactive isotope or a fluorescent marker conjugated to the light chain. According to some embodiments, the light chain is a recombinantly produced protein transfected to the target cell by a protein transfection reagent or incorporation of a protein transduction peptide or peptido-mimetic.

According to some embodiments, the light chain is a fusion protein comprising a light chain fused with a protein marker, preferably a fluorescent protein marker. In some embodiments, the fluorescent marker is green fluorescent protein. In some embodiments, the cell comprises a gene that encodes a fusion protein comprising the light chain and a protein marker, preferably a fluorescent protein marker. In some embodiments, the fluorescent marker is green fluorescent protein. When the gene is expressed, the fusion protein comprising the light chain and marker protein is produced in the cell.

According to some embodiments, the localization pattern is determined using various imaging/microscopy techniques, including confocal microscopy. In other embodiments, autoradiography can be used. In some embodiments, the localization pattern is determined by automated computer-assisted scanning. For examining membranes that do not colocalize with VAMP (e.g., Golgi, ER), a fluorescence probe for specific organelles or organellar membranes may be advantageously employed. In some embodiment, the movement of the LC may be detected using these organelle markers.

Enzyme Activity

According to some embodiments, the invention relates to methods of identifying compounds that alters, e.g., inhibit or enhances, the enzymatic activity of Clostridial toxin. The methods comprise a test enzymatic assay that comprise contacting a sample containing the light chain of the toxin with an enzymatic substrate of the light chain in the presence of the test compound and determining whether the substrate is processed by the light chain into enzymatic product. The absence of processing of the enzymatic substrate into enzymatic product indicates that the test compound inhibits enzymatic activity. Conversely, the enhancement of processing of the enzymatic substrate into enzymatic product indicates that the test compound enhances enzymatic activity.

According to some embodiments, the Clostridial toxin is selected from the group consisting of: the toxin that is produced by Clostridium beratti, the toxin that is produced by Clostridium butyricum, the toxin that is produced by Clostridium tetani bacterium and the toxin that is produced by Clostridium botulinum. In some embodiments, the Clostridial toxin is selected from the group of Clostridial toxins consisting of: botulinum toxin types A, B, C₁, D, E, F and G.

According to some embodiments, the method further comprises performing a negative control enzymatic assay. Such a negative assay comprises the steps of: contacting a sample that comprises the light chain (e.g., the entire BoNT or just the light chain) with an enzymatic substrate in the presence of an enzymatic assay negative control compound or no added compound and determining whether the enzymatic substrate is processed by the light chain into enzymatic product. The absence of processing of the enzymatic substrate into enzymatic product indicates that, for the test compound, results demonstrating processing of the substrate into product in this enzymatic assay are inclusive.

According to some embodiments, the method further comprises performing a positive control enzymatic assay. Such a positive control assay comprises the steps of contacting a sample that comprises the light chain (e.g. the entire BoNT or just the light chain) with an enzymatic substrate in the presence of an enzymatic assay positive control compound and determining whether the enzymatic substrate is processed by the light chain into enzymatic product. For an activity-enhancing compound, the processing of the enzymatic substrate into enzymatic product indicates that, for the test compound, results demonstrating processing of the substrate into product in this assay are inclusive. For an inhibitory compound, the absence of processing of the enzymatic substrate into enzymatic product indicates that, for the test compound, results demonstrating a lack of processing of the substrate into product in this assay are inclusive.

According to some embodiments, multiple test enzymatic assays are performed in which different concentrations of test compound are used in individual test assays. Generally, in such embodiments, the test compound is used in step-wise 10-fold dilutions to ascertain its activity and the concentration necessary to observe activity.

According to some embodiments, the method is performed using duplicate assays. By using duplicate assays, results are more reliable. In the event that one of the duplicate test assays is positive and the corresponding duplicate produces negative results, the assay is repeated.

According to some embodiments, the light chain is Botulinum type A or E and the enzymatic substrate is synaptosomal associated protein (SNAP-25) According to some embodiments, the light chain is Botulinum toxin types B, D, F or G or tetanus toxin and the enzymatic substrate is vesicle-associated protein (VAMP, also called synaptobrevin) and/or cellubrevin. According to some embodiments, the light chain is botulinum toxin type C₁ and the enzymatic substrate is syntaxin or SNAP-25.

According to some embodiments, the determination of whether the enzymatic substrate has been processed into the enzymatic product is determined by Western blot using cell lysates and antibodies against substrate or product. According to some the embodiments, the determination of whether the enzymatic substrate has been processed into the enzymatic product is determined by an ELISA assay. In some embodiments, antibodies are used which bind to the enzymatic substrate but not the enzymatic products. In other embodiments, antibodies are used which bind to the enzymatic products but not the enzymatic substrate. In some embodiments, antibodies are used which bind to the enzymatic substrate (and also optionally to the enzymatic product). In some embodiments, fluorescence assays like GFP-SNAP or FRET assays may be employed.

Dual Assay Methods

According to some embodiments, test compounds are tested in both the test localization assay and the test enzymatic assay as described above. In some embodiments, the test localization assay is performed prior to the test enzymatic assay. In some embodiments, the test compound is tested in the test enzymatic assay prior to testing it in the test localization assay. In some embodiments, the assays are run independently.

Compound Formulations and Salts

The compounds of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The compounds of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions.

The term “pharmaceutically acceptable salts” refers to physiologically and pharmaceutically acceptable salts of the compounds of the invention: i.e., salts that retain the desired biological persistence of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

Methods of Altering Biological Persistence in a Mammal

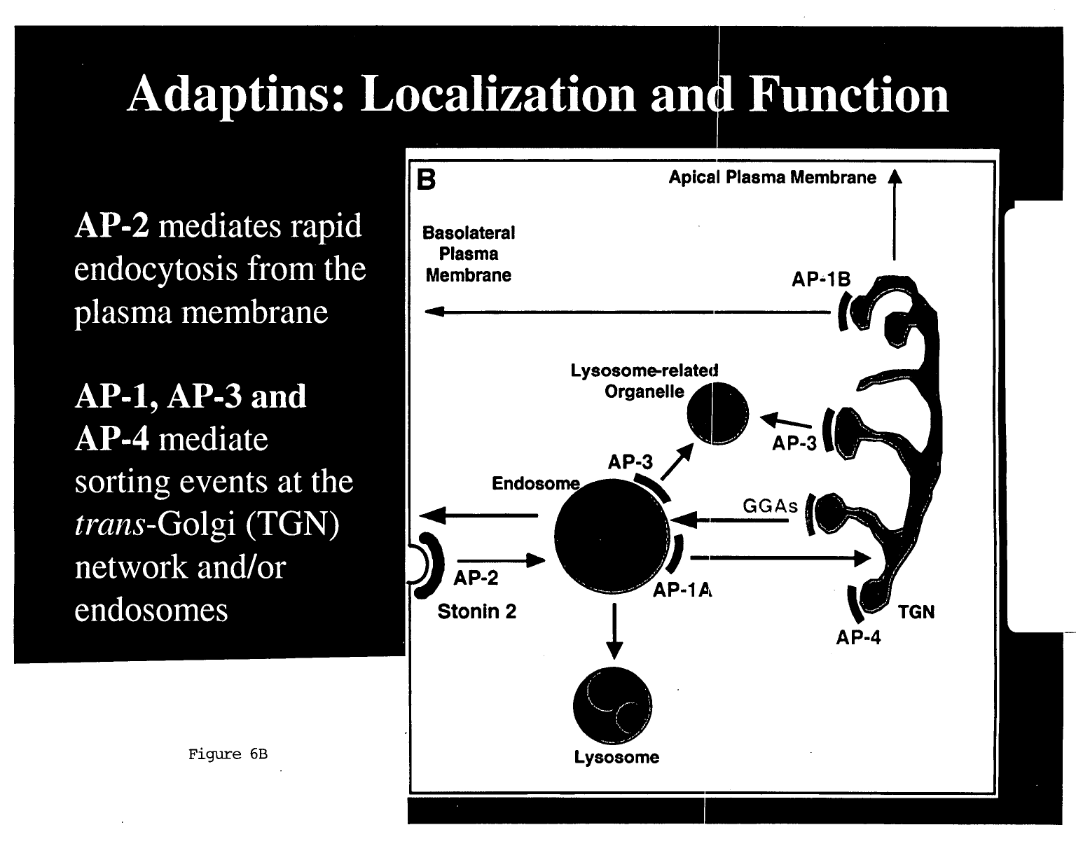

The present invention also features methods for altering the biological persistence of a Clostridial toxin, e.g., botulinum toxin in a mammal, e.g., humans. The methods comprise the step of administering to the mammal a therapeutically effective amount of the compound to either reduce or increase the biological persistence of Clostridial toxin, e.g., botulinum toxin. Some non-limiting examples of compounds that may reduce the biological persistence by altering localization are small peptides or peptidomimetics similar to the L chain C-terminal di-leucine motif, the N-terminal 8 amino acids of the L chain, the regions on the beta-subunit of the adaptin (AP) proteins or the VHS subunit of GGAS that are involved in intracellular trafficking. See FIGS. 6A and 6B

In some embodiments, a compound of the present invention is administered to the mammal before the mammal is exposed to the Clostridial toxin. In some embodiments, a compound of the present invention is administered to the mammal the same time that the mammal is exposed to the Clostridial toxin. In some embodiments, a compound of the present invention is administered to the mammal after the mammal is exposed to the Clostridial toxin.

In some embodiments, a compound of the present invention is administered to reduce the biological persistence of botulinum toxin in a mammal. For example, in cases of accidental or deliberate botulinum toxin intoxication, a compound of the present invention may be administered to alleviate or treat the intoxication. In some embodiments, the compound of the present invention reduces the biological persistence of botulinum toxins by more than 25%, preferably more than 50%, more preferably more than 75%, for example 90%. In some embodiments, the compound of the present invention reduces the biological persistence of botulinum toxin type A by more than 25%, preferably more than 50%, more preferably more than 75%, for example 90%. In some embodiments, the compound of the present invention reduces the biological persistence of botulinum toxin type B by more than 25%, preferably more than 50%, more preferably more than 75%, for example 90%.

In some embodiments, a compound of the present invention is administered to increase the biological persistence of a botulinum toxin. For example, botulinum toxin types B or E may be used as therapeutics. However, types B and E have short biological persistence, relative to type A. In some embodiments, type B and/or E toxin is administered along with a compound of the present invention. The compound may be administered before, simultaneously with or subsequently to the administration of botulinum toxin type B and/or E. In some embodiments, the administration of the compounds of the present invention increase the biological persistence of botulinum toxin types B and/or E by more than about 1.5 to 2 fold, preferably 4 fold, more preferably 8 fold, or, for example, 10 fold.

Of course, an ordinarily skilled medical provider can determine the appropriate dose and frequency of administration(s) to achieve an optimum clinical result. That is, one of ordinary skill in medicine would be able to administer the appropriate amount of the compound of the present invention at the appropriate time(s) to effectively treat a condition. The dose of the compounds to be administered depends upon a variety of factors, including the severity of the condition.

The compounds of the present invention may be administered through any effective route. For example, the compounds of the present invention may be administered intramuscularly, orally, systemically, and/or locally. Although examples of routes of administration and dosage are provided, the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14^{th} edition, published by McGraw Hill).

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Subcellular Localization of LC/A, LC/B and LC/E in Neuronal Cells

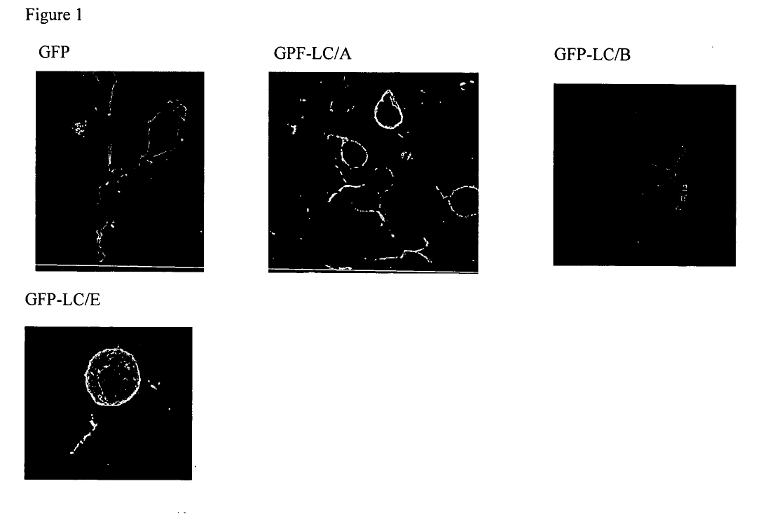

In order to study the subcellular localization of the light chain from serotypes A, B and E, plasmids encoding light chain sequences fused to Green Fluorescent Protein (GFP) were constructed. Plasmids expressing GFP-LC/A, GFP-LC/B and GFP-LC/E were transfected into PC12 cells and, 24 hours later, cells were differentiated into a neuronal phenotype with NGF. FIG. 1 shows confocal images of the transfected and differentiated PC12 cells. LC/A clearly localizes to the plasma membrane, LC/B is dispersed throughout the cell including the nucleus, and LC/E is cytoplasmic with substantial nuclear exclusion.

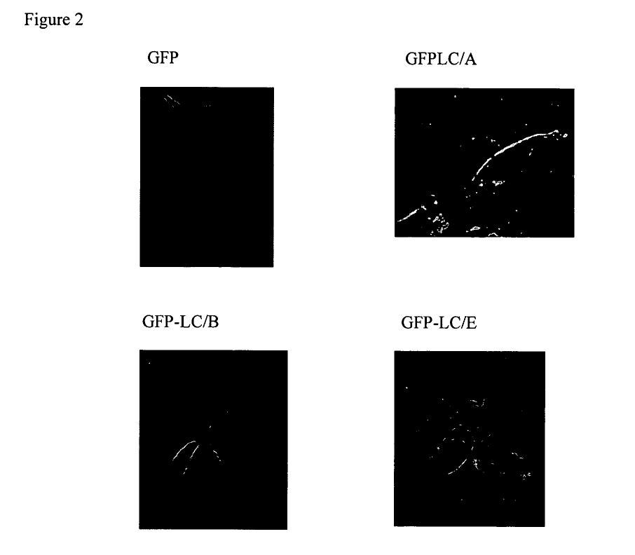

The localization of the LC proteins is identical in the human neuroblastomna SH-SY5Y (as shown in FIG. 2), and in the insulinoma cell line HIT-T15. These insulinoma cells secrete insulin using the same SNAREs as in neuronal cells. These data confirm that the subcellular localization defined in PC12 cells extends to other exocytotic cell lines of neuronal and non-neuronal origin. Moreover, the localization of LCs is similar, but not identical, in other non-neuronal cells that do not contain SNAP25, as observed for HeLa and HEK293.

### Example 2

### Identification of Sequences and Motifs in LC/A Important For Plasma Membrane Localization.

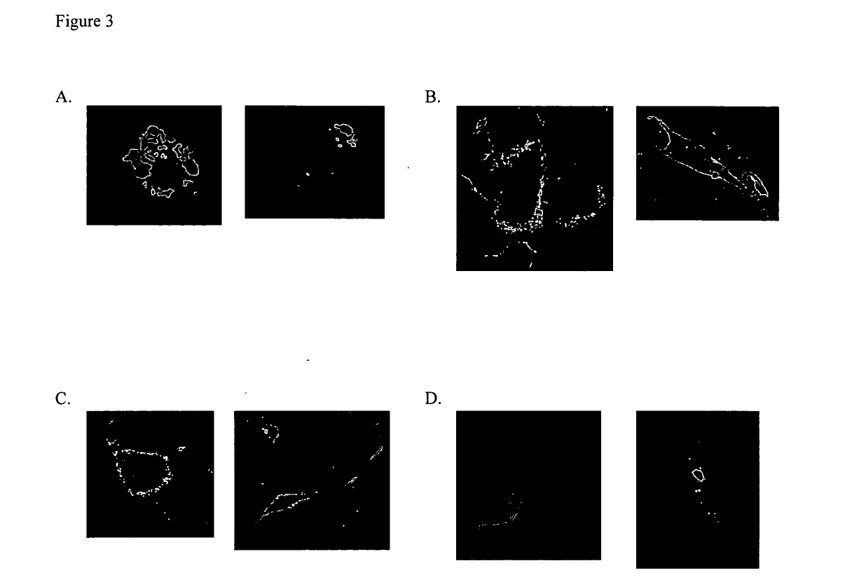

A truncated form of LC/A (Tyr9-Leu415) has been reported as the minimal essential domain of the endoprotease, retaining a diminished catalytic activity towards SNAP25 (Kadkhodayan et al, 2000, Protein Expr Purif 19, 125-30). The truncated LC/A structure (PDB structure # 1E1H—this structure is deposited in the database (see www.rcsb.org/pdb/ as of Oct. 7, 2003) was reported to be similar to the LC/A structure in the holotoxin. A GFP fusion protein, GFP-LC/A (ΔN8ΔC22), containing the same truncations, was prepared and fluorescence imaging revealed the protein to no longer be localized to the plasma membrane. Instead, fluorescence appeared in perinuclear structures as well as throughout the cytoplasm (FIG. 3A). These data suggest that signals involved in directing LC/A to the plasma membrane may reside within the deleted regions. Analysis of the primary sequence of LC/A using motif databases did reveal consensus sequences for common glycosylation and phosphorylation sites, in addition to the zinc-endopeptidase motif characteristic of Botulinum neurotoxins. Further analysis of the sequences revealed the presence of a putative di-Leucine motif (D/ExxxLL—SEQ ID NO:1) at the C-terminus of LC that was only present in the BoNT/A serotype (FEFYKLL—SEQ ID NO:2). Mutation of the leucines into alanines, or mutation of the acidic residue (D/E-4 with respect to the first leucine) has been shown to disrupt the motif and thereby affect interaction with AP adaptors, protein internalization, and/or intracellular localization. 1E1H is the crystal structure of rLC/A(ΔN8/ΔC22). The peptide backbone of the crystal structure of this truncated form of rLC/A overlays very well on the backbone of LC/A in the holotoxin (150 kD-HC/LC) structure that is known. This suggests that the truncated LC is not substantially misfolded as a result of the truncation. It also suggests that LC structures do not change substantially once dissociated from the HC.

A di-leucine to di-alanine mutant (L427A and L428A), GFP-LC/A(AA), was prepared and expressed in PC12 cells. Analysis of the localization of the protein product revealed that disruption of the di-leucine motif caused changes in the distribution of LC/A at the plasma membrane. A large percentage of cells showed a more diffuse distribution of GFP-LC/A(AA) at the plasma membrane, suggesting that the protein can reach the target membrane but may be loosely anchored (FIG. 3B). A smaller percentage of cells revealed an even more diffuse cytoplasmic localization of GFP-LC/A(AA).

An almost identical pattern of localization (FIG. 3C) was found when a truncated construct, GFP-LC/A(ΔC22), lacking 22 C-terminal amino acids was expressed, suggesting that the primary localization signal present at the C-terminus is the di-leucine motif. Since neither truncation (ΔC22) nor mutation (LL->AA) of the C-terminus produced the same effect as the double truncation mutant (ΔN8ΔC22), it was postulated that LC/A localization signals for plasma membrane targeting were also present at the N-terminus of the protein or the N-terminus has structural importance for localization.

A truncated LC/A in which eight amino acids were removed from the N-terminus (GFP-LC/A(ΔN8)) was expressed in differentiated PC12 cells. Fluorescence visualization of this mutant revealed a complete loss of plasma membrane localization (FIG. 3D). GFP fluorescence appeared dispersed throughout the cytoplasm with nuclear exclusion. In approximately 5% of these cells, a localization similar to that of the GFP-LC/A (ΔN8/ΔC22) mutant could be seen.

These data support the presence of signals or structural elements important for plasma membrane localization in the N-terminus of LC/A. It is also possible that the signal, whether sequence or structural, may be located elsewhere in the protein and is affected by the removal of the N-terminus.

### Example 3

### Mutations of the LC/A That Affect Localization Also Affect Catalytic Activity

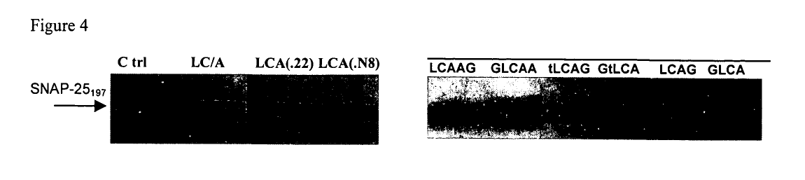

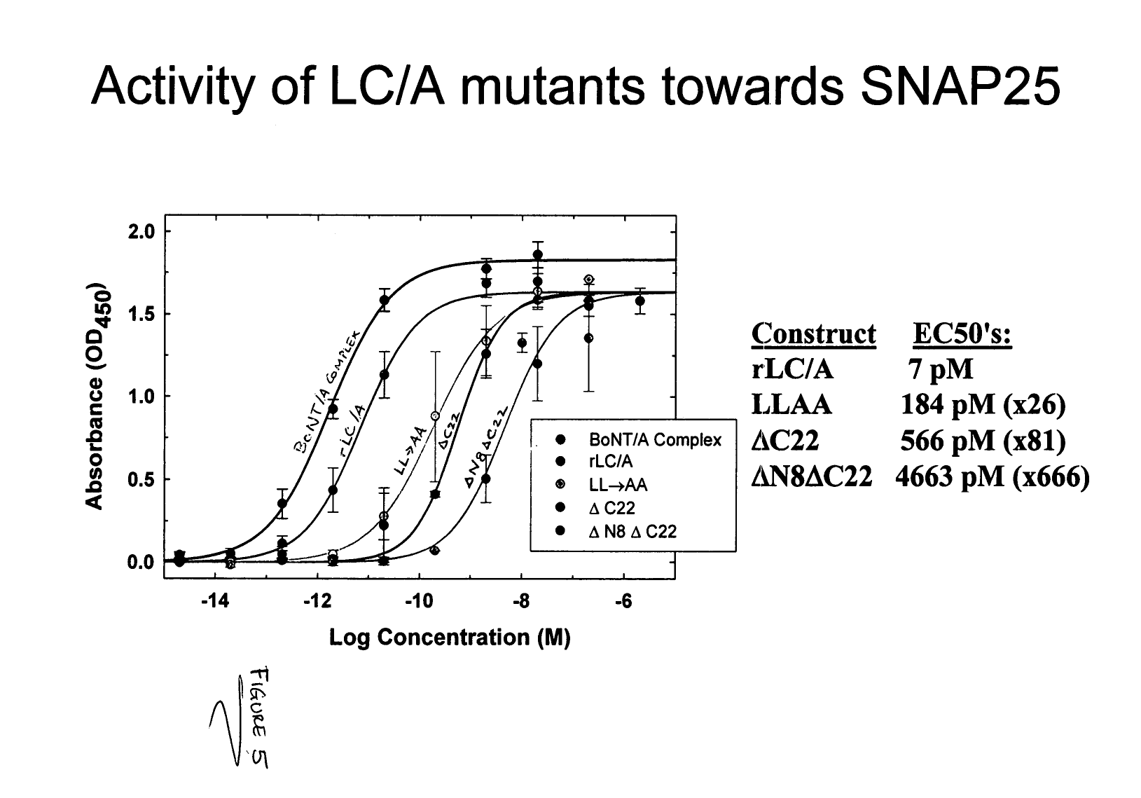

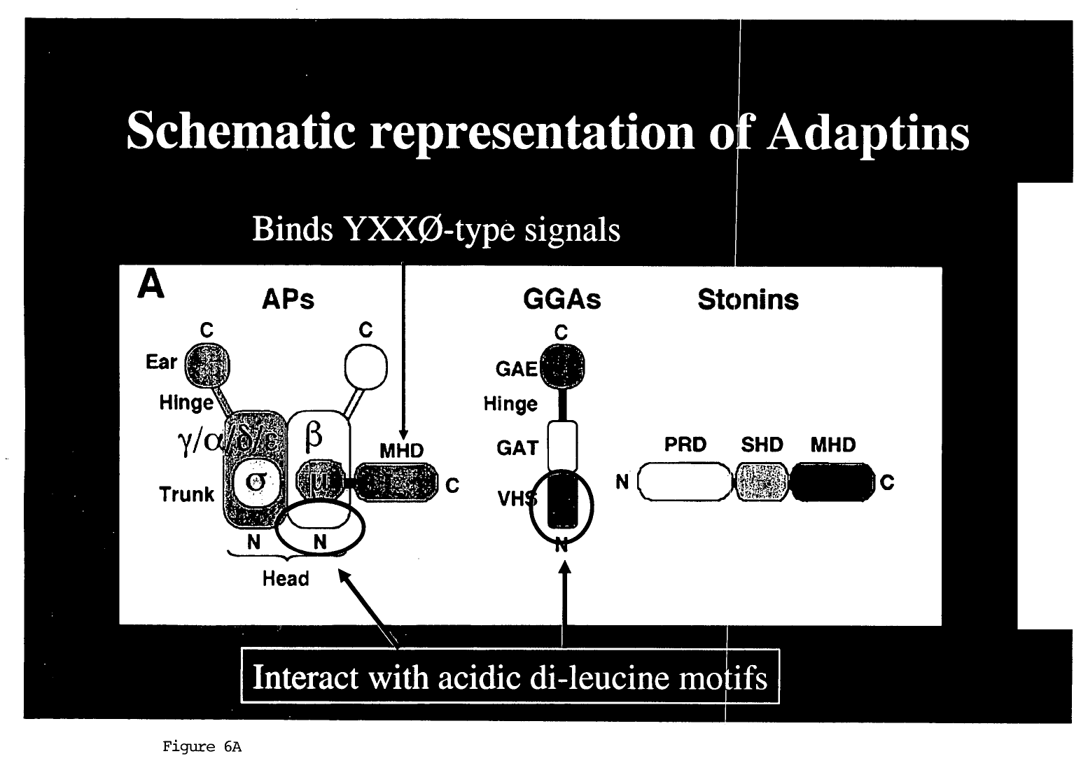

In order to evaluate the catalytic activity of the LC/A mutants described above, cell lysates from transfected cells were subjected to Western blot analysis using an antibody that specifically recognizes SNAP25₁₉₇, the product of LC/A cleavage. FIG. 4 shows that the GFP-LC/A(AA) and the GFP-LC/A(ΔC22) mutants seem to have a slightly lower level of activity. The GFP-LC/A(ΔN8) mutant that localizes to the cytoplasm is less active than the wt LC/A, and the double truncated mutant GFP-LC/A (ΔN8/ΔC22) has only residual activity.

Transient transfection analysis cannot be used to quantitate differences in catalytic activity due to the variability within samples intrinsic to the technique. In addition, small differences in enzymatic activity may not be detectable in these cellular analyses, since the amount of protein expressed is likely higher than the amount of protein taken up during toxin treatment. To further characterize their proteolytic activities, LC/A mutants were expressed in E. coli as polyhistidine-tagged fusion proteins. Protease activity was analyzed with an ELISA assay using a biotinylated substrate corresponding to SNAP25₁₃₄₋₂₀₆ and a SNAP25₁₉₇-specific antibody. The most extensive truncation of LC/A at both the N- and C-terminus (ΔN8/ΔC22) clearly diminished the activity (666-fold less active than wild-type). A deletion of the C-terminal 22 amino acids of the light chain, rLC/A(ΔC22), is 81-fold less active than wild-type LC/A. Interestingly, rLC/A(AA) mutation is 26-fold less active than wild-type rLC/A (FIG. 5). The activities of LC/A mutants towards SNAP25 are as follows: the EC₅₀ of rLC/A is 7 pM, LLAA is 184 pM, ΔC22 is 566 pM, and ΔN8/ΔC22 is 4663 pM. With respect to localization, rLC/A localizes to the plasma membrane, regardless of the position of the GFP-tag. rLC/A(AA) and rLC/A(ΔC22) diffuse throughout the plasma membrane. rLC/A(ΔN8) is mainly in the cytoplasm and rLC/A(ΔN8/ΔC22) is dispersed intracellularly.

### Example 4

### Design of Compounds That Inhibits the Biological Persistence of Toxins

Structure-based compound (drug) design methods involve modeling the three-dimensional structure of a protein, e.g., botulinum toxin, that is a potential drug target interacting with various lead compounds to guide drug discovery. A lead compound is a small molecule, a peptide, or a peptidomimetic that serves as the starting point for an optimization involving many small molecules that are closely related in structure to the lead compound. Further, potential compounds are modeled computationally to estimate their “fit” to the target by computing a scoring function or an energy function.

The algorithms used in drug design consider both structural and functional interactions, such as steric fit, hydrogen bonding and hydrophobic interactions. The initial design phase of a drug is usually followed by the synthesis of the lead compound, target protein binding assays, and co-crystallization of the compound and target for x-ray structural studies. Empirical information regarding how the lead compound actually binds to the target drives the refinement of the lead compound to improve target-binding. The refined lead compound is then synthesized and complexed with the target, and further refined in a reiteractive optimization process.

The disclosures in patent application Ser. Nos. 09/620,840; 09/910,346; and 10/136,106 and disclosures herein show that a mutation to the leucine-based motif and/or a tyrosine based motif on the light chain disrupts localization and enzymatic activity of a toxin, for example BoNT/A. Thus, the leucine-based motif and/or the tyrosine-based motif regions of the toxin would be a possible site for the design of a compound which would disrupt localization and/or enzymatic activities of a toxin, for example BoNT/A. For example, based on the three-dimensional structure of the toxin at the leucine-based and/or tyrosine-based regions of the protein, a lead compound may be designed and synthesized. This lead compound may be a small molecule, a peptide, or a peptidomimetic that would serve as the starting point for an optimization involving many small molecules that are closely related in structure to the lead compound. The various compounds may be screened according to the present invention for biological activities.

Other compounds which may be tested in accordance with the present invention include the molecules disclosed by Burnett et al. in the article entitled “Novel small molecule inhibitors of botulinum neurotoxin A metalloprotease activity”, Biochemical and Biophysical Research Communications 310 (2003) 84-93, the disclosure of which is incorporated in its entirety herein by reference.

### Example 5

### Screening of Compounds

As discussed herein, the subcellular localization patterns of the L chains from Botulinum toxin types A, B and E are distinctly different, with the LC/A localizing to the plasma membrane, the LC/E localizing to the cytoplasm (with nuclear exclusion), and the LC/B being dispersed throughout the cell (including the nucleus). Further, it is believed that the duration of action of the neurotoxins are related to their subcellular localization. For example, the LC of BoNT/A localizes to the plasma membrane, a compartment known for its low rate of protein recycling. This specific localization may protect the LC/A from proteolytic degradation within neurons. Thus, a compound which disrupts the localization of the LC of BoNT/A would reduce the biological persistence of BoNT/A.

Compounds could be first screened using recombinant LCs in vitro for inhibition of activity using an ELISA assay or newly developed GFP/SNAP assay or FRET assays (See U.S. patent Application Ser. Nos. 2003143651 and 2003020948, and Allergan's patent application Ser. No. 10/163,106 the disclosures of which are incorporated in their entirety herein by reference). Interactions of the compounds with the recombinant LCs will also be tested by measuring binding of the two molecules in vitro. Compounds will be tested in stable cell lines expressing different GFP-LC constructs. Changes in LC localization, half-life, and activity can be assessed. In addition, a cell-based assay to test BoNT activity measuring inhibition of neurotransmitter release in SH-SY5Y cells has been developed. Toxin can be applied to the cells in combination with the compounds, or compounds can be added at a later time point, and inhibition of exocytosis relative to toxin only controls will be measured. Duration of neurotoxin effect will also be evaluated. Potential active compounds would ultimately be tested in animal models of toxin activity: DAS (Digit Abduction Score), LD₅₀ and muscle atrophy. Top candidates will be transferred to toxicology for their analysis.

### Example 6

### Exemplary Methods For Identifying Compounds That Alter the Biological Persistence of a Clostridial Toxin

In some embodiments, the method of identifying a compound that alters a biological persistence of a Clostridial toxin comprises performing a test localization assay. In some embodiments, the test localization assay comprises the steps of:

(a) contacting a cell that comprises a Clostridial toxin light chain with a test compound,

(b) observing a localization pattern of the light chain in the cell following contacting the cell with the test compound,

(c) comparing the determined localization pattern to a localization pattern of a light chain in a cell in an absence of the test compound, and

(d) identifying a test compound that alters a biological persistence of a Clostridial toxin by determining a change in the localization pattern of the light chain in the cell following contacting the cell with the test compound.

In some embodiments, the test compounds are found to decrease or inhibit the biological persistence of Botulinum toxin type A. In some embodiments, the test compound is capable of disrupting the localization pattern of type A. For example, the compound causes the light chain of Botulinum toxin type A to not localize at the plasma membrane anymore, or causes the localization to be different as compared to the control. In some embodiments, compounds that decrease biological persistence of Botulinum toxin type A causes the light chain of type A to be dispersed throughout the cell. In some embodiments, the localization pattern of a light chain of botulinum toxin type A in a cell in the presence of the test compound is less localized to the plasma membrane than the localization pattern of a light chain in a cell in an absence of the test compound. For example, the density of the light chain of toxin type A that is localized to the plasma membrane is reduced by more than about 20%, preferably more than about 40%, more preferably more than about 60%, for example 80%.

In some embodiments, the test compounds are found to increase the biological persistence of Botulinum toxin type F. In some embodiments, the test compounds are found to increase the biological persistence of Botulinum toxin types B and E. In some embodiments, the test compound is capable of causing the Botulinum toxin type B to localize to the plasma membrane or other more relevant membranes for type B, e.g., TGN, Goldi, ER (VAMP is a v-SNARE inserted in the membrane of neurotransmitter containing vesicles). In some embodiments, the test compound is capable of causing the Botulinum toxin type B to localize to a cellular membrane providing protection from degradation in the cell. In some embodiments, the test compound is capable of causing the Botulinum toxin type E to localize to the plasma membrane in a pattern similar to that of Botulinum toxin type A localization. Without wishing to limit the invention to any theory or mechanism of operation, it is believed that the localization of type B or type E to the relevant membranes (e.g., for type B: TGN, Goldi, ER) or plasma membrane similarly to that of type A enhances biological persistence.

### Example 7

### Methods of Producing Clostridial Toxins For Use in Screening Assays of Compounds

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C₁ are apparently produced as only a 700 kD or 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain non-toxin hemaglutinin proteins and a non-toxin and non-toxic nonhemaglutinin protein. These-non-toxin proteins (which along with the botulinum toxin molecule comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, and norepinephrine (Habermann E., et al., Tetanus Toxin and Botulinum A and C Neurotoxins Inhibit Noradrenaline Release From Cultured Mouse Brain, J Neurochem 51(2);522-527:1988); CGRP, substance P and glutamate (Sanchez-Prieto, J., et al., Botulinum Toxin A Blocks Glutamate Exocytosis From Guinea Pig Cerebral Cortical Synaptosomes, Eur J. Biochem 165;675-681:1897). Thus, when adequate concentrations are used, stimulus-evoked release of most neurotransmitters is blocked by botulinum toxin. (See e.g. Pearce, L. B., Pharmacologic Characterization of Botulinum Toxin For Basic Science and Medicine, Toxicon 35(9);1373-1412 at 1393; Bigalke H., et al., Botulinum A Neurotoxin Inhibits Non-Cholinergic Synaptic Transmission in Mouse Spinal Cord Neurons in Culture, Brain Research 360;318-324:1985; Habermann E., Inhibition by Tetanus and Botulinum A Toxin of the release of [³H]Noradrenaline and [³H]GABA From Rat Brain Homogenate, Experientia 44;224-226:1988, Bigalke H., et al., Tetanus Toxin and Botulinum A Toxin Inhibit Release and Uptake of Various Transmitters, as Studied with Particulate Preparations From Rat Brain and Spinal Cord, Naunyn-Schmiedeberg's Arch Pharmacol 316;244-251:1981, and; Jankovic J. et al., Therapy With Botulinum Toxin, Marcel Dekker, Inc., (1994), page 5).

Botulinum toxin type A can be obtained by establishing and growing cultures of Clostridium botulinum in a fermenter and then harvesting and purifying the fermented mixture in accordance with known procedures. All the botulinum toxin serotypes are initially synthesized as (substantially) inactive single chain proteins that must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically not activated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level.

High quality crystalline botulinum toxin type A can be produced from the Hall A strain of Clostridium botulinum with characteristics of ≧3×10⁷ U/mg, an A₂₆₀/A₂₇₈ of less than 0.60 and a distinct pattern of banding on gel electrophoresis. The known Schantz process can be used to obtain crystalline botulinum toxin type A, as set forth in Schantz, E. J., et al, Properties and use of Botulinum toxin and Other Microbial Neurotoxins in Medicine, Microbiol Rev. 56;80-99:1992. Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×10⁸ LD₅₀ U/mg or greater; purified botulinum toxin type B with an approximately 156 kD molecular weight with a specific potency of 1-2×10⁸ LD₅₀ U/mg or greater, and; purified botulinum toxin type F with an approximately 155 kD molecular weight with a specific potency of 1-2×10⁷ LD₅₀ U/mg or greater.

Botulinum toxins and/or botulinum toxin complexes can be obtained from List Biological Laboratories, Inc., Campbell, Calif.; the Centre for Applied Microbiology and Research, Porton Down, U.K.; Wako (Osaka, Japan), Metabiologics (Madison, Wis.) as well as from Sigma Chemicals of St Louis, Mo. Pure botulinum toxin can also be used to prepare a pharmaceutical composition.

As with enzymes generally, the biological activities of the botulinum toxins (which are intracellular endopeptidases) is dependant, at least in part, upon their three dimensional conformation. Thus, botulinum toxin type A is detoxified by heat, various chemicals, surface stretching and surface drying. Additionally, it is known that dilution of the toxin complex obtained by the known culturing, fermentation and purification to the much, much lower toxin concentrations used for pharmaceutical composition formulation results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the toxin may be used months or years after the toxin containing pharmaceutical composition is formulated, the toxin can be stabilized with a stabilizing agent such as albumin and gelatin.

A commercially available botulinum toxin containing pharmaceutical composition is sold under the trademark BOTOX® (available from Allergan, Inc., of Irvine, Calif.). BOTOX® consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein, associated hemagglutinin proteins and NTNH. The crystalline complex is re-dissolved in a solution containing saline and albumin and sterile filtered (0.2 microns) prior to vacuum-drying. The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin type A purified neurotoxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX® can be stored in a refrigerator at about 2° C. to about 8° C. Reconstituted, refrigerated BOTOX® has been reported to retain its potency for at least about two weeks. Neurology, 48:249-53:1997.

### Example 8

### Release Assay on SH-SY5Y Cells

Materials

SH-SY5Y cells maintenance media: EMEM(Gibco 11090):F12(Gibco 11765-054) 1:1, 2 mM glutamine, 0.1 mM NEAA, 10% FBS, 10 mM HEPES, 1× penicillin/streptomycin;

Matrixgel coated 24-well tissue culture plate (VWR 62405-103);

l-(7,8-³H)-Noradrenaline (Amersham TRK-584);

Ready Protein Scintillation cocktail (Beckman 586604);

BSS (0.44 mM KH2PO4, 1.2 mM MgC12, 2 mM CaCl₂, 4.2 mM NaHCO3, 20 mM HEPES, 137 mM NaCl, 5 mM KCl, 5 mM D-glucose);

BSS loading buffer: BSS+0.2 mM ascorbic acid and 0.2 mM pargyline;

100 mM K+release buffer: 51 mM NaCl, 100 mM KCl, 0.5 mM MgCl₂, 2.2 mM CaCL₂, 5.6 mM D-glucose, 15 mM HEPES, pH 7.4 and filtration;

Steps:

Cells are grown in a T75 flask until passage 20 (cell line was at passage 10 at the time of purchase from ECACC);

8 days before release assay, cells are plated at 2×10⁵ cells/well in 24-well Matrixgel coated plates;

Toxin is added at day 5 at various concentrations, then cells are incubated for 3 days;

Cells are washed with 500 μl of 2× with BSS+0.2 mM Ascorbic, 0.2 mM pargyline;

Cells are loaded with ³H-noradrenaline at concentration of 2 μCi/ml in the same buffer above at 37° C. for 1 h;

After loading, cells are washed 4×15 min with BSS at 37C in the water bath and appropriate release buffers 500 μl/well for 5 min at 37° C., for each condition are tested in triplicate;

The buffer is removed to fresh, chilled 24-well plate, and cells are centrifuged at 1200 RPM, for 4 min at 4° C.;

400 μp of release buffer is removed to a scintillation vial;

In parallel, 500 μl Trypsin/EDTA is added to the cells, which are held at room temperature for 5 min;

250 μl is removed to a scintillation vial, 5 ml of Ready Protein scintillation fluid is added to each vial, and vials are shaken vigorously before a scintillation reading is taken.

Results are recorded based on the following formula: % release=DPM release/DPM release+DPM remaining.

### Example 9

### Cell-Based Assays

The present assay is to establish a cell-based assay to evaluate neurotoxin activity in formulated samples (BOTOX®), bulk toxin, and in process samples. In addition, this assay will be useful in determining and comparing the IC50 of new and modified toxins using cell culture as a tool.

In contrast to current ELISA assay, the cell-based assay will cover all three steps in toxin action: binding, internalization/translocation, and protease activity, providing a better measure of toxin activity. The ideal cell line for this assay will be one that will allow measurement of binding to the high affinity receptor, translocation and activity in cleaving SNAP25. Since the receptor for BoNT/A is unknown, it is necessary to identify wild type cell lines expressing the high affinity of receptor to bind BoNT/A. It is known that BoNTs bind to motor neurons and spinal cord neurons with the highest affinity, they also bind to DRGs, cortical neurons and hippocampal neurons with high affinity. Due to the fact that primary cells are impractical for such an assay, it is preferred that a cell line from neuronal tissue such as spinal cord, hippocampal or cortex is identified. The second kind of cell lines that would be potentially useful is “neuronal-like” cells that are known to uptake toxin such as SH-SY5Y and NG108-C 15.

Table 1 lists possible useful cell lines. Not many neuronal cell lines have been Clostridial neurotoxin uptake, and in some cases the serotype tested was not Neuronal cell lines originated from spinal cord, cortex, hippocampus and DRGs considered.

Neuroblastoma SH-SY5Y cell line was selected as reference to compare the results.

TABLE 1
Commercial and Academic Cell Lines information

| | |
|---|---|
| Commercial Cell Lines | Non-commercial Cell Lines |

| Name | Origin | Name | Origin |
|---|---|---|---|
| SK-N-DZ | Human Neuroblastoma | LA-N-2 | Human |
| | | | Neuroblastoma |
| SK-N-F1 | Human Neuroblastoma | | Mouse Motor |
| | | | Neuron |
| SK-N-SH | Human Neuroblastoma | NSC-19 | Mouse Motor |
| | | | Neuron |
| SH-SY5Y | Human Neuroblastoma | SC | Human Spinal Cord |
| SK-N-BE(2) | Human Neuroblastoma | CNS | Human Central |
| | | | Nervous System |
| BE(2)-C | Human Neuroblastoma | CNh | Murine Cerebral |
| | | | Cortex |
| Neuro 2a | Mouse Neuroblastoma | M4b | Murine Spinal Cord |
| NB4 1A3 | Mouse Neuroblastoma | G4b | Murine Dorsal Root |
| | | | Ganglia |
| N1E-115 | Mouse Neuroblastoma | HN-33 | Murine |
| | | | hippocampal/ |
| | | | neuroblastoma |
| | | | hybrid |
| HT-22 | Mouse Hippocampal | | |
| NG108-15 | Mouse/Rat Neuroblastoma | | |
| HCN-1A | Human cortical neuron | | |
| HCN-2 | Human cortical neuron | | |
| ND8 | neuroblastoma/DRG hybrid | | |
| ND7 | neuroblastoma/DRG hybrid | | |
| CRL-7947 | Human Spinal Cord | | |

Methods:

Western Blot Analysis:

During preliminary screening, it was determined that only SH-SY5Y and PC12 cells uptake ³H-noradrenaline and release it upon stimulation, but the rest of cell lines tested did not either uptake this neurotransmitter or they release it a higher levels in basal buffer. To determine if cells can be intoxicated by BoNT/A, a Western Blot method to screen the cell lines for toxin uptake by using anti-SNAP25₁₉₇ (Zymed #1 antiserum) antibody that only recognizes the cleaved SNAP25 product was developed.

Cells were plated in Matrigel or poly-D-lysine/Laminin coated 6-well plates for 24 hours. Then BoNT/A at different concentrations was added to the cells in the culture medium over night. Cells were collected in 15 ml Falcon tubes, washed once with 1 ml of PBS, and then transferred to 1.5 ml microcentrifuge tubes. Cells were lysed in 0.5 ml of lysis buffer (50 mM HEPES, 150 mM NaCl, 1.5 mM MgCl₂, 1 mM EGTA, 10% glycerol and 1% triton X-100) on rotator at 4° C. for 1 hour. Lysed cells were spun down at 5000 rpm at 4° C. for 10 min to eliminate debris; supernatants were transferred to fresh siliconized tubes. Protein concentrations were measured by the Braford's method; the protein was resuspended in 1× sample buffer at 1 mg/ml concentration. Samples were boiled for 5 min, 30 μl (30 μg/lane) of the samples were loaded on 4-12% Tris-HCl gels. Proteins were transferred to PVDF membranes, and blocked in 5% non-fat milk in TBST buffer for 1 hour at room temperature. The cleaved SNAP25 was detected with anti-SNAP25₁₉₇ antibody diluted in blocking buffer; blot was washed extensively, and the bound antibody was detected with horseradish peroxidase conjugated to species-specific antibody. After final washes the membrane was reacted with SuperSignal western blot detection reagents and exposed to the film. For example, the Typhoon 4000 Imager (Amersham) or a suitable similar instrument is used and may be more sensitive than photographic film exposure.

Radiolabeled Neurotransmitter Release Assay:

Induced secretion of ³H-noradrenaline was used to measure inhibition of exocytosis by toxin as the screening method. SH-SY5Y cells under passage 25 were routinely cultured in T75 flask, plated at 2×10⁵ cells per well in 24-well Matrigel thin layer coated plates. Five days after plating, cells were treated for 3 days with different concentrations of toxin in culture medium. Cells were washed twice with 1 ml of loading buffer (BSS: 0.44 mM KH₂PO₄, 1.2 mM MgCl₂, 2 mM CaCl₂, 4.2 mM NaHCO₃, 20 mM HEPES, 137 mM NaCl, 5 mM KC1 and 5 mM D-glucose, pH 7.4 plus 0.2 mM ascorbic acid and 0.2 mM pargyline) and loaded with 0.5 ml of ³H-noradrenaline (2 μCi/ml) in the loading buffer for 1 hour in the 37° C. incubator. After washing three times with BSS (3×15 min), the basal cell release was measured using 0.5 ml of BSS buffer, followed by a measurement of induced release by incubating the cells with 0.5 ml of BSS with 100 mM KCl and 45 mM NaCl as stimulated buffer. The 24-well tissue culture plates were centrifuged to pellet floating cells, then 0.2 ml of supernatants were transferred to 24-well scintillation plates to count. The remaining cells in the plate were lysed in 0.5 ml of 2 M acetic acid plus 0.1% trifluoroacetic acid, then 0.2 ml were transferred to be counted. Release was expressed as a percentage of the total cellular counts in basal buffer prior to determination of release, and percentage inhibition was calculated relative to non-intoxicated cells.

Results:

Evaluation of the SH-SY5Y Cell Line:

SH-SY5Y cells were grown for 5 days after plating and then treated for an additional 3 days with BoNT/A (either pure toxin or 900 KDa complex). Cells showed some inhibition of release at concentrations of less than 1 nM and reached maximum inhibition around 20 nM. The IC₅₀ determined by SigmaPlot software was about 1 nM. However, the cells were not healthy after three days of toxin treatment at concentrations greater than 15 nM, cells started peeling off from the bottom of the plate.

Western blot analysis showed dose-dependent cleavage of SNAP25 by BoNT/A. The detection of the cleaved SNAP25 at concentrations of BoNT/A complex at 0.5 nM was possible. The Western blot time course study showed the SH-SY5Y cells require an incubation of overnight (approximately 16 hours) or up to 24 hours for toxin uptake. Further time course studies would be advantageous in order to better define the minimal incubation time between 4 hours and overnight.

Cell Lines Screening:

In order to screen the cells for toxin uptake, the remaining cell lines (Table 1) were screened using the Western Blot analysis method that detects SNAP25 cleavage.

Cells were cultured in 6-well Matrigel coated plates. BoNT/A complex was added to the cells and incubated overnight at concentrations of 1.67 nM. Cells were then washed with 1×PBS, removed from the plate and pelleted. Cells were lysed in the lysis buffer at 14° C. for 1 hour, and then spun down at 5000 rpm for 10 min. Total protein concentration was measured after discarding the debris. 30 μg of total protein per lane was loaded per cell line. The rank order for efficient toxin uptake by the various experimental groups was determined to be:

Neuro-2a>SH-SY5Y>NG108-15>NIE115>SK-N-DZ=SK-N-F1>BE (2)-C>SK-N-SH=NB4 1A3.

NG108-C15 cells were evaluated for toxin binding using avidin-biotinylated BoNT/C₁ (Noriko Yokosawa et al.). The neurotoxin bound with high efficiency to NG108-C15, but bound poorly to human neuroblastoma cell lines. In our study, it was shown that this cell line can uptake pure BoNT/A (150 kD). Cleaved SNAP25 was detected at 0.33 nM using the Typhoon instrument, and at 0.67 nM by Western blot analysis.

Evaluation of the Neuro-2a Cell Line:

SNAP25 cleavage can be detected with overnight treatments as low as 0.3 nM of BoNT/A complex in the Neuro-2a cell line. It is possible that the limit of detection could be lower than 0.3 nM, the lowest dose tested.

### CONCLUSIONS

Neuro-2a is the best one of the ten neuroblastoma cell lines tested in uptake of botulinum toxin type A as screened by Western blot analysis. This cell line takes up toxin at concentrations as low as 0.3 nM. SH-SYSY is the only cell line that takes ³H-noradrenaline and releases it upon Ca²⁺/K⁺ stimulation. The approximate IC50 is 1-2 nM. The Neuro-2a cell line in the present conditions is not sensitive enough to measure a single vial of Botox®.

### Example 10

### Cell-Based Assay to Measure Neurotoxin Activity

A cell-based assay will be able to measure binding of the neurotoxin to its receptor, internalization, translocation, and protease activity (each of these are determined indirectly by measuring the conversion of substrate to product by the protease). Identification of cell lines with the high affinity uptake for toxin is the hallmark for the development of a cell-based assay. Botulinum toxin serotypes A, B, C I, C2, D, E, F, or G or derivatives thereof may be used in these assays. Neuro-2a is the preferred cell line for toxin uptake as screened using Western blot analysis. Neuro-2a cells internalize BoNT/A complex (900 kDa) at concentration as low as 0.3 nM via a functional pathway. It is known that specific gangliosides must be present at the plasma membrane of the target cells along with an unidentified protein receptor for high affinity uptake of BoNT/A into cells.

Western Blot Analysis:

The neurotoxin treatment and Western Blot method developed to screen cell lines for toxin uptake using polyclonal antibody, Zymed # 1, were adopted to detect cleaved SNAP25. Briefly cells were plated in poly-D-lysine/Laminin coated 6-well plates for 24 hours. BoNT/A (900 KDa complex) was added to the cells at different concentrations in the culture medium over night. Cells were collected in 15 ml Falcon tubes, washed once with 1 ml of PBS, and then transferred to 1.5 ml microcentrifuge tubes. Cells were lysed in 0.5 ml of lysis buffer (50 mM HEPES, 150 mM NaCl, 1.5 mM MgCl₂, 1 mM EGTA, 10% glycerol and 1% triton X-100) on rotator at 4° C. for 1 hour. Lysed cells were spun down at 5000 rpm at 4° C. for 10 min to eliminate debris; supernatants were transferred to fresh siliconized tubes. Protein concentrations were measured by the Bradford method and resuspended in 1× SDS sample buffer at 1 mg/ml or higher concentration. Samples were boiled for 5 min, 40 μl of the samples were loaded on 4-12% Tris-HCl gels. Proteins were transferred to PVDF membranes, and blocked in 5% non-fat milk in TBST buffer for 1 hour at room temperature. The cleaved SNAP25 was detected with anti-SNAP25₁₉₇ antibody diluted in blocking buffer; blot was washed extensively, and the bound antibody was detected with horseradish peroxidase conjugated to species-specific secondary antibody.

A Typhoon 9410 Imager (Amersham) was used for Western Blot Analysis instead of traditional photographic film. After the final washes, the membrane was reacted with ECL Plus western blot detection reagent (Amersham), and the blot was incubated at room temperature for 5 min to develop. The choice of pixel size and PMT voltage settings will depend on the individual blot. Membranes were scanned and quantified using Typhoon Scanner and Imager Analysis software.

Radiolabeled Neurotransmitter Release Assay:

Cell lines under passage 25 were routinely cultured in T75 flask. For the assay, cells were plated at 2×10⁵ cells per well in 24-well Matrigel thin layer coated plates. After 5 days incubation, cells were washed twice with 1 ml of loading buffer (BSS: 0.44 mM KH₂PO₄, 1.2 mM MgCl₂, 2 mM CaCl₂, 4.2 mM NaHCO₃, 20 mM HEPES, 137 mM NaCl, 5 mM KCl and 5 mM D-glucose, pH 7.4 plus 0.2 mM ascorbic acid and 0.2 mM pargyline) and loaded with 0.5 ml of ³H-labeled neurotransmitters (noradrenaline, glycine or 5HT) at 2 μCi/ml in the loading buffer for 2 hours in the 37° C., 5% CO₂ incubator. After washing three times with BSS (3×10 min), the basal cell release was measured using 0.5 ml of BSS buffer (5.44 mM K⁺), followed by a measurement of induced release by incubating the cells with 0.5 ml of BSS with 100 mM KCl and 45 mM NaCl as depolarization buffer. The 24-well tissue culture plates were centrifuged to pellet floating cells, then 0.2 ml of supernatants were transferred to 24-well scintillation plates to count. The remaining cells in the plate were lysed in 0.5 ml of 2 M acetic acid plus 0.1% trifluoroacetic acid, then 0.2 ml were transferred to be counted. Release was expressed as a percentage of the total cellular counts in basal buffer prior to determination of release, and percentage inhibition was calculated relative to non-intoxicated cells.

GT1b Ganglioside Treatment:

Cells were cultured in the Poly-D-lysine/Laminin coated 6-well plates for Western blot assay or 24-well for release assay in the culture medium with 10% FCS for 24 hours. GT1b (Alexis, San Diego, Calif.) was dissolved in PBS at 5 mg/ml as stock solution and stored at −20° C. At the time of treatment, GT1b was diluted in serum free medium at 25 μg/ml and added to cells. Cells were incubated at 37° C., 7.5% CO₂ for 24 hours. GT1b containing medium was removed and BoNT/A complex was added at various concentrations in regular culture medium containing 10% FBS and was incubated over night.

The results showed that cleavage of SNAP25 is detected in SH-SY5Y cells as early as 6 hours after treatment with 1 nM BoNT/A complex.

A similar time course study was conducted using the Neuro-2A cell line. Since Neuro-2A is more sensitive to toxin uptake than SH-SY5Y, a study using shorter exposure times for this cell line was performed. The BoNT/A complex at 1 nM concentration was added to cells and cells were collected at 0, 10, 20, 30, 60 min, and 16 hours (the typical overnight incubation). Cleavage of SNAP25 was detected by Western blot and visualized with the Typhoon Imaging system as early as 10 min after exposure. This result indicates that Neuro-2A cell line binds toxin through a high affinity uptake system.

Ganglioside GT1b treatment:

To determine if the addition of exogenous gangliosides can increase the sensitivity of cell lines to BoNT/A, SH-SY5Y and Neuro-2A cells were plated in culture medium in 6-well plates for 24 hours. The day before the assay, culture medium was replaced with 25 μg/ml GT1b in serum-free medium or with serum-free medium alone as a negative control. Preliminary results showed that ganglioside GT1b increases the amount of detectable cleaved SNAP25 by approximately two folds when Neuro-2A cells were incubated with 0.25 nM and 0.5 nM of BoNT/A complex. No cleaved SNAP25 was observed in cells with GT 1b only (no toxin) or without treatment.

Because GT-1b increased toxin uptake, and loading more protein for the Western blot improved the sensitivity of detection of cleaved SNAP25 when using lower doses of BoNT/A, we performed ganglioside treatments on Neuro-2a cells with 25 μg/ml of GT1b, using lower doses of BoNT/A. As expected, GT1b treatment significantly increased SNAP25 cleavage (approx. 2 fold) and facilitated detection of cleaved product following an overnight treatment with 12.5 pM BoNT/A complex. In the absence of GT1b, SNAP25₁₉₇ is nearly undetectable after treatment with 12.5 pM of BoNT/A. The dose limit for detection of SNAP25₁₉₇ in Neuro-2A cells containing exogenous gangliosides is approximately 10 pM for BoNT/A. If one vial of Botox® containing approximately 5 ng of is diluted in 0.5 mL, the toxin concentration will be 10 pM. Neuro-2a cells seem to have suitable sensitivity for the development of a cell-based assay for BoNT/A.

Treatments with GT-1b were also performed on the SH-SY5Y cells. The presence of GT1b decreased the detection limit of BoNT/A by 50% (to 0.25 nM) based on detection of cleaved SNA25₁₉₇. For the following study, the amount of total protein loaded per lane was increased from 40 μg to 140 μg. This allowed the detection of cleaved SNAP25₁₉₇ at 0.125 nM of BoNT/A complex treatment with or without GT1b treatment; however, cleavage was more readily observable with treatment. It appears that SH-SY5Y cells do not have a high affinity system for toxin uptake.

IC50 Determination by Release Assay and Western Blot:

The IC50 of inhibition of neurotransmitter release by BoNT/A (pure and complex) using SH-SY5Y cells in the ³H-NA release assay can be calculated. This release assay indirectly measures binding, internalization, translocation, and protease activity by measuring exocytosis. The SH-SY5Y cell line only expresses the low affinity uptake system for the toxin. The IC50 for BoNT/A complex was 1.7 nM on SH-SY5Y cells, suggesting that this cell line may be useful for testing bulk toxin and in-process samples.

The antibody SMI-81 is able to detect both the intact and neurotoxin cleaved SNAP25. Removal of nine amino acids of the BoNT/A complex produces a sufficient molecular weight change for the two bands to be resolved. However, the process described in the literature requires a Methanol-Chloroform extraction that does not allow protein quantitation. The protocol using samples extracted in regular lysis buffers was optimized. Quantification was carried out using Typhoon image analysis software. The amount of cleavage was expressed as a percentage of the total cleaved and uncleaved SNAP25 bands. A preliminary experiment was performed on Neuro-2a cells, treated overnight with six concentrations of BoNT/A complex up to 5 nM. At these concentrations, cleavage of SNAP25 did not reach 100%. Further experiments can use a broader range of BoNT/A doses for determination of the IC50 for toxin. In addition, it has been reported that 35% cleavage of SNAP-25 at the neuromuscular junction induces full paralysis.

### CONCLUSIONS

Because in the Neuro-2A cell line BoNT/A can be internalized and produce detectable levels of product within a few minutes following treatment with low pM BoNT/A, Neuro-2A cells appear to contain a high affinity receptor for BoNT/A. Addition of exogenous ganglioside GT1 b to neuroblastoma cell lines increases toxin internalization. The product cleaved by BoNT/A was detected with toxin treatments as low as 12 pM in Neuro-2a cells containing exogenous GT1b. GT1b treatment doubled the amount of cleaved SNAP25 detected after toxin treatment.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

What is claimed is:

1. A method of identifying a compound that alters a biological persistence of a Clostridial toxin, the method comprising performing a test localization assay having the steps of:
(a) contacting a cell that comprises a Clostridial toxin light chain with a test compound,
(b) observing a localization pattern of the light chain in the cell following contacting the cell with the test compound,
(c) comparing the observed localization pattern to a localization pattern of a light chain in a cell in an absence of the test compound, and
(d) identifying a test compound that alters a biological persistence of a Clostridial toxin by determining a change in the localization pattern of the light chain in the cell following contacting the cell with the test compound.

2. The method of claim 1 wherein the Clostridial toxin is a botulinum toxin type A.

3. The method of claim 2 wherein the biological persistence of the botulinum toxin type A is reduced.

4. The method of claim 3 wherein the localization pattern of a light chain of botulinum toxin type A in a cell in the presence of the test compound is less localized to the plasma membrane than the localization pattern of a light chain in a cell in an absence of the test compound.

5. The method of claim 1 further comprising performing a negative control localization assay that comprises the steps of:
(a) contacting a cell that comprises the light chain with a localization assay negative control compound; and
(b) determining whether the localization pattern of the light chain in the cell differs following contacting the cell with the localization assay negative control compound compared to the localization pattern of the light chain in the cell in the absence of the localization assay negative control compound,
wherein a change in the localization pattern of the light chain in the cell following contacting the cell with the localization assay negative control compound indicates that the test localization assay results are inconclusive.

6. The method of claim 1 further comprising performing a positive control localization assay that comprises the steps of:
(a) contacting a cell that comprises the light chain with a localization assay positive control compound, wherein the localization assay positive control compound is known to change the localization pattern of the light chain in a cell when contacted with a cell that comprises the toxin; and
(b) determining whether the localization pattern of the light chain in the cell differs following contacting the cell with the localization assay positive control compound compared to the localization pattern of the light chain in the cell in the absence of the localization assay positive control compound,
wherein an absence of change in the localization pattern of the light chain in the cell following contacting the cell with the localization assay positive control compound indicates that the test localization assay results are inconclusive.

7. The method of claim 1 comprising multiple test localization assays wherein individual test assays are performed using different concentrations of test compound.

8. The method of claim 1 comprising performing at least a duplicate test localization assays.

9. The method of claim 1 wherein the cell is selected from the group consisting of: Neuro-2A cells, PC₁₂ cells, SHSY-5Y cells, HIT-T15 cells, HeLa cells, HEK293 cells, and primary and established neuronal culture cells from spinal cord, cortex, hippocampus and dorsal root ganglion.

10. The method of claim 1 wherein the cell comprises a gene that encodes the light chain, which is expressed to produce the light chain in the cell.

11. The method of claim 1 wherein the toxin is contacted with the cell in an amount effective to be taken up by the cell and produce an identifiable localization pattern of the light chain in the cell.

12. The method of claim 1 wherein the light chain is labelled.

13. The method of claim 1 wherein the light chain is labeled with a radio-active isotope or a fluorescent marker.

14. The method of claim 1 wherein the light chain is expressed as a fusion protein comprising a light chain fused with a fluorescent marker.

15. The method of claim 1 wherein the localization pattern is determined using microscopic techniques that allow for the analysis of changes in subcellular localization, including confocal microscopic systems.

16. The method of claim 1 further comprising a test enzymatic assay that comprises:
(a) contacting a sample containing the light chain with an enzymatic substrate of the light chain in the presence of the test compound; and
(b) determining whether the substrate is processed by the light chain into enzymatic product;
wherein the absence of processing of the enzymatic substrate into enzymatic product indicates that the test compound inhibits enzymatic activity, and the enhancement of processing of the enzymatic substrate into enzymatic product indicates that the test compound enhances enzymatic activity.

17. The method of claim 16 further comprising performing a negative control enzymatic assay that comprises the steps of:
(a) contacting a sample that comprises the light chain with an enzymatic substrate in the presence of an enzymatic assay negative control compound or no added compound; and
(b) determining whether the enzymatic substrate is processed by the light chain into enzymatic product;
wherein the absence of processing of the enzymatic substrate into enzymatic product indicates that test enzymatic assay results are inclusive.

18. The method of claim 16 further comprising performing a positive control enzymatic assay that comprises the steps of:
(a) contacting a sample that comprises the light chain with an enzymatic substrate in the presence of an enzymatic assay positive control compound; and
(b) determining whether the enzymatic substrate is processed by the light chain into enzymatic product;
wherein processing of the enzymatic substrate into enzymatic product indicates that test enzymatic assay results are inclusive.

19. The method of claim 16 comprising multiple test enzymatic assays wherein individual enzymatic test assays are performed using different concentrations of test compound.

20. The method of claim 16 comprising performing at least duplicate test enzymatic assays.

21. The method of claim 16 wherein the light chain is from Botulinum types A, B, C₁, D, E, F or G or Tetanus toxin, and the enzymatic substrate is a synaptosomal associated protein, SNAP25, VAMP, cellubrevin, syntaxin or fragments, peptides, peptidomimetics, or mixtures thereof.

22. The method of claim 16 wherein the processing of it into enzymatic product is determined by Western blot, ELISA assay, GFP-SNAP assay, FRET assay, or a combination of said assays, using an antibody that specifically binds to uncleaved enzymatic substrate and/or enzymatic products.

23. A method of identifying a compound that inhibits the enzymatic activity of Clostridial toxin comprising a test enzymatic assay that comprises:
(a) contacting a sample containing a light chain of a Clostridial toxin with an enzymatic substrate of the light chain in the presence of the test compound; and
(b) determining whether the substrate is processed by the light chain into enzymatic product;
wherein the absence of processing of the enzymatic substrate into enzymatic product indicates that the test compound inhibits enzymatic activity, and the enhancement of processing of the enzymatic substrate into enzymatic product indicates that the test compound enhances enzymatic activity.

24. The method of claim 23 wherein the Clostridium toxin is produced by Clostridium beratti, Clostridium butyricum, Clostridium tetani or Clostridium botulinum.

25. The method of claim 1 wherein Clostridial toxin is selected from the group consisting of: botulinum toxin types A, B, C₁, D, E, F and G.

26. The method of claim 23 wherein the Clostridial toxin is botulinum toxin type A.

27. The method of claim 23 further comprising performing a negative control enzymatic assay that comprises the steps of:
(a) contacting a sample that comprises the light chain with an enzymatic substrate in the presence of an enzymatic assay negative control compound or no added compound; and
(b) determining whether the enzymatic substrate is processed by the light chain into enzymatic product;
wherein the absence of processing of the enzymatic substrate into enzymatic product indicates that test enzymatic assay results are inclusive.

28. The method of claim 23 further comprising performing a positive control enzymatic assay that comprises the steps of:
(a) contacting a sample that comprises the light chain with an enzymatic substrate in the presence of an enzymatic assay positive control compound; and
(b) determining whether the enzymatic substrate is processed by the light chain into enzymatic product;
wherein processing of the enzymatic substrate into enzymatic product indicates that test enzymatic assay results are inclusive.

29. The method of claim 23 comprising multiple test enzymatic assays wherein individual enzymatic test assays are performed using different concentrations of test compound.

30. The method of claim 23 comprising performing at least a duplicate test enzymatic assays.

31. The method of claim 23 wherein the light chain is from Botulinum types A, B, C₁, D, E, F or G or Tetanus toxin, and the enzymatic substrate is a synaptosomal associated protein, SNAP25, VAMP, cellubrevin, syntaxin or fragments, peptides, peptidomimetics, or mixtures thereof.

32. The method of claim 23 wherein the processing of it into enzymatic product is determined by Western blot, ELISA assay, GFP-SNAP assay, FRET assay, or a combination of said assays, using an antibody that specifically binds to uncleaved enzymatic substrate and/or enzymatic products.

33. The method of claim 23 further comprising performing a test localization assay that comprises the steps of:
(a) contacting a cell that comprises a Clostridial toxin light chain with a test compound; and
(b) determining whether the localization pattern of the light chain in the cell differs following contacting the cell with the test compound compared to the localization pattern of the light chain in the cell in the absence of the test compound,
wherein a change in the localization pattern of the light chain in the cell following contacting the cell with the test compound indicates that the test compound alters the biological persistence of the toxin.

34. The method of claim 33 further comprising performing a negative control localization assay that comprises the steps of:
(a) contacting a cell that comprises the light chain with a localization assay negative control compound; and
(b) determining whether the localization pattern of the light chain in the cell differs following contacting the cell with the localization assay negative control compound compared to the localization pattern of the light chain in the cell in the absence of the localization assay negative control compound,
wherein a change in the localization pattern of the light chain in the cell following contacting the cell with the localization assay negative control compound indicates that the test localization assay results are inconclusive.

35. The method of claim 33 further comprising performing a positive control localization assay that comprises the steps of:
(a) contacting a cell that comprises the light chain with a localization assay positive control compound, wherein the localization assay positive control compound is known to change the localization pattern of the light chain in a cell when contacted with a cell that comprises the toxin; and
(b) determining whether the localization pattern of the light chain in the cell differs following contacting the cell with the localization assay positive control compound compared to the localization pattern of the light chain in the cell in the absence of the localization assay positive control compound,
wherein an absence of change in the localization pattern of the light chain in the cell following contacting the cell with the localization assay positive control compound indicates that the test localization assay results are inconclusive.

36. The method of claim 33 comprising multiple test localization assays wherein individual test assays are performed using different concentrations of test compound.

37. The method of claim 33 comprising performing duplicate test localization assays.

38. The method of claim 33 wherein the cell is selected from the group consisting of: Neuro-2A cells, PC₁₂ cells, SH-SY5Y cells, HIT-T15 cells, HeLa cells, HEK293 cells, and primary and established neuronal culture cells from spinal cord, cortex, hippocampus and dorsal root ganglion.

39. The method of claim 33 wherein the cell comprises a gene that encodes the light chain, which is expressed to produce the light chain in the cell.

40. The method of claim 33 wherein the toxin is contacted with the cell in an amount effective to be taken up by the cell and produce an identifiable localization pattern of the light chain in the cell.

41. The method of claim 33 wherein the light chain used in the assay is labeled.

42. The method of claim 33 wherein the light chain used in the assay is labeled with a radio-active isotope or a fluorescent marker.

43. The method of claim 33 wherein the light chain used in the assay is expressed as a fusion protein comprising a light chain fused with a fluorescent marker.

44. The method of claim 33 wherein the localization pattern is determined using microscopic techniques that allow for analysis of changes in subcellular localization, including confocal microscopy.

45. A method of identifying a compound that inhibits the biological persistence of a Clostridial toxin comprising performing a test localization assay that comprises the steps of:
(a) contacting a cell that comprises a Clostridial toxin light chain with a test compound; and
(b) determining whether the localization pattern of the light chain in the cell differs following contacting the cell with the test compound compared to the localization pattern of the light chain in the cell in the absence of the test compound,
wherein a change in the localization pattern of the light chain in the cell following contacting the cell with the test compound indicates that the test compound inhibits the biological persistence of the toxin.

46. The method of claim 45 wherein the Clostridial toxin is produced by Clostridium beratti, Clostridium butyricum, Clostridium tetani or Clostridium botulinum.

47. The method of claim 45 wherein Clostridial toxin is selected from the group consisting of: botulinum toxin types A, B, C₁, D, E, F and G.

48. The method of claim 45 wherein the Clostridial toxin is botulinum toxin type A.

49. A method for altering a biological persistence of a Clostridial toxin in a mammal, the method comprising the step of administering to the mammal a test compound, wherein the test compound is identified by performing a test localization assay having the steps of:
(a) contacting a cell that comprises a Clostridial toxin light chain with a test compound,
(b) observing a localization pattern of the light chain in the cell following contacting the cell with the test compound,
(c) comparing the determined localization pattern to a localization pattern of a light chain in a cell in an absence of the test compound, and
(d) identifying a test compound that alters a biological persistence of a Clostridial toxin by determining a change in the localization pattern of the light chain in the cell following contacting the cell with the test compound.

50. The method of claim 49 wherein the compound is administered before the mammal is exposed to Clostridial toxin.

51. The method of claim 49 wherein the compound is administered during the time the mammal is exposed to Clostridial toxin.

52. The method of claim 49 wherein the compound is administered after the mammal is exposed to Clostridial toxin.

53. The method of claim 49 wherein the compound reduces the biological persistence of Clostridial toxin in the mammal.

54. The method of claim 49 wherein the compound increases the biological persistence of the Clostridial toxin in the mammal.

55. The method of claim 53 wherein the Clostridial toxin is a botulinum toxin type A.
